(19) United States
(12) Patent Application Publication
(10) Pub. No.: US 2024/0285328 A1
(43) Pub. Date: Aug. 29, 2024

(54) CRYOTHERAPY DEVICE

(71) Applicant: Vessi Medical Ltd., Mobile Post Misgav (IL)

(72) Inventors: Eyal KOCHAVI, Haifa (IL); Mordechai SIMCHON, Haifa (IL)

(73) Assignee: Vessi Medical Ltd., Mobile Post Misgav (IL)

(21) Appl. No.: 18/560,417

(22) PCT Filed: May 12, 2022
(86) PCT No.: PCT/IL2022/050496

§ 371 Date: Nov. 13, 2023

Related U.S. Application Data
(60) Provisional application No. 63/188,015, filed on May 13, 2021.

(51) Int. Cl. A61B 18/02; A61B 1/05; A61B 18/00

(52) U.S. Cl. CPC A61B 18/0218; A61B 1/05; A61B 2018/0097; A61B 2018/0212

(57) ABSTRACT

A cryotherapy system, including, an elongated device having a body, a proximal end and a distal end, configured to be positioned inside a hollow organ, including:
a cryo path fluidically connecting a cryogenic fluid source with the distal end of the device, wherein the cryo path is configured to allow cryogenic fluid flow into the hollow organ;
an optics assembly inside the body configured to visualize a field of view between the device distal end and a target region inside the hollow organ;
a washing path comprising at least one washing opening at the device distal end, wherein the washing path connects a washing fluid source with the at least one washing opening and configured to release washing fluid within the hollow organ;
at least one flow reducer within the washing path configured to narrow an inner lumen of the washing path through which the washing fluid flows.

### RELATED APPLICATION

This application claims the benefit of priority under 35 USC § 119(e) of U.S. Provisional Patent Application No. 63/188,015 filed 13 May 2021, the contents of which are incorporated herein by reference in their entirety.

This application is related to International Application Number PCT/IL2018/050124 filed on 4 Feb. 2018, the contents of which are all incorporated by reference as if fully set forth herein in their entirety.

### FIELD AND BACKGROUND OF THE INVENTION

The present invention, in some embodiments thereof, relates to a cryotherapy device and, more particularly, but not exclusively, to a cryotherapy device for treating hollow organ diseases.

### SUMMARY OF THE INVENTION

Some examples of some embodiments of the invention listed below may include features from more than one example and/or fewer than all features of an example:

Example 1. A cryotherapy system, comprising,
an elongated cryotherapy device having an elongated body, a proximal end and a distal end, wherein said device is shaped and sized to be positioned inside a hollow organ, comprising:
a cryo inflow path inside said elongated body fluidically connecting a cryogenic fluid source with said distal end of said cryotherapy device, wherein said cryo inflow path is configured to allow cryogenic flow from said cryogenic fluid source into said hollow organ;
an optics assembly having a distal end inside said elongated body configured to visualize a field of view between said device distal end and a target region inside said hollow organ;
a washing inflow path inside said elongated body comprising at least one washing opening at said device distal end, wherein said washing inflow path fluidically connects a washing fluid source with said at least one washing opening and configured to release washing fluid within said hollow organ;
at least one flow reducer positioned within said washing inflow path shaped and sized to narrow an inner lumen of said washing inflow path through which said washing fluid flows.
   Example 2. A system according to example 1, wherein said at least one flow reducer blocks at least 10% of an inner lumen of said washing inflow path.
   Example 3. A system according to any one of examples 1 or 2, wherein said at least one opening is positioned in a wall of said washing inflow path to aim washing fluid towards said optics assembly in said distal end and/or towards a field of view (FOV) distal to said optics assembly.
   Example 4. A system according to example 3, wherein said at least one flow reducer is located distally to said at least one washing opening, and is configured to increase washing fluid pressure within a portion of said washing inflow path comprising said at least one opening.
   Example 5. A system according to example 3, wherein said at least one flow reducer is positioned within a portion of said washing inflow path comprises said at least one washing opening.
   Example 6. A system according to example 5, wherein said at least one flow reducer comprises at least one channel or aligned openings forming a channel, in said at least one flow reducer in a fluid communication with said at least one opening, configured to direct washing fluid through said at least one flow reducer to said at least one washing opening or to suck fluid from said hollow organ into said washing inflow path.
   Example 7. A system according to any one of examples 3 to 6, wherein said washing inflow path comprises at least one distal washing opening at a distal end of said washing inflow path configured to release washing fluid into a treatment space within said hollow organ located distally to said elongated cryotherapy device.
   Example 8. A system according to example 7, wherein said at least one flow reducer is positioned within said washing inflow path proximally to said at least one distal washing opening.
   Example 9. A system according to example 7, wherein said at least one distal washing opening is an opening in said at least one flow reducer.
   Example 10. A system according to any one of examples 7 to 9, wherein said cryo inflow path comprises at least one distal opening configured to release cryofluid into said treatment space and/or towards a treatment target within the hollow organ, and wherein said at least one washing opening surrounds at least partly said cryo inflow path distal opening.
   Example 11. A system according to any one of examples 3 to 10, wherein said at least one flow reducer contacts an inner surface of said washing inflow path and/or an outer surface of said cryo inflow path.
   Example 12. A system according to example 11, wherein said at least one flow reducer is shaped as a ring, an arc contacting the inner surface of said washing inflow path and/or an outer surface of said cryo inflow path.
   Example 13. A system according to example 11, wherein said at least one flow reducer comprises a protrusion extending from said inner surface of said washing inflow path and/or said outer surface of said cryo inflow path.
   Example 14. A system according to any one of examples 3 to 13, wherein said at least one opening comprises a plurality of openings axially and/or angularly distributed in a portion of a wall of said washing inflow path facing said optics assembly and/or said FOV.
   Example 15. A system according to any one of examples 1 to 13, wherein said at least one opening comprises a plurality of openings axially and/or angularly distributed in a portion of a wall of said washing inflow path at least partly surrounding said washing inflow path inner lumen.
   Example 16. A system according to any one of examples 14 or 15, wherein said plurality of openings have different shapes and/or sizes.
   Example 17. A system according to any one of examples 14 to 16, wherein said plurality of openings are evenly distributed in said wall portion.
   Example 18. A system according to any one of examples 14 to 17, wherein said plurality of openings are distributed in varying densities of openings per area in said wall portion.
   Example 19. A system according to example 1, wherein said at least one flow reducer is located proximally to said at least one washing opening, and is configured to increase flow speed of washing fluid exiting through said at least one washing opening into the hollow organ.
   Example 20. A system according to example 1, wherein said at least one flow reducer is located at a distal end of said washing fluid inflow path and comprises one or more openings which are aligned with said at least one washing opening to allow passage of washing fluid through said one or more flow reducer openings into said at least one washing opening.
   Example 21. A system according to any one of the previous examples, wherein said cryotherapy device comprises at least one fluid evacuation flow path inside said elongated body having at least one first evacuation opening at a distal end of said at least one fluid evacuation flow path, and at least one second evacuation opening in a circumference of a wall of said evacuation flow path, wherein said second evacuation opening is located proximally to said first evacuation opening;
and wherein said at least one fluid evacuation flow path is configured to remove fluid and/or particles from said hollow organ through said at least one first evacuation opening and/or through said at least one second evacuation opening.
   Example 22. A system according to example 21, wherein said cryotherapy device comprises at least one evacuation seal located within said at least one fluid evacuation flow path between said at least one first evacuation opening and said at least one second evacuation opening, and configured to block at least 10% of evacuation fluid flow through said at least one first evacuation opening.
   Example 23. A system according to example 22, wherein a distal end of said optics assembly is positioned distally to said second evacuation opening, and wherein said at least one evacuation seal is positioned between said optics assembly distal end and said at least one second evacuation opening.
   Example 24. A system according to any one of examples 21 to 23, comprising an outer sleeve surrounding said device, and wherein said at least one second evacuation opening is an opening in said outer sleeve or is in a fluid communication with at least one opening in said outer sleeve.
   Example 25. A system according to any one of the previous examples, wherein said at least one washing opening has a maximal width or diameter in a range of 0.01 mm-2 mm.
   Example 26. A system according to any one of the previous examples, wherein said cryo inflow path and said washing inflow path are coaxial along at least 30% of a length of said washing inflow path.
   Example 27. A system according to any one of examples 1 to 25, wherein said washing inflow path surrounds at least partly said cryo inflow path along at least 30% of a length of said cryo inflow path.
   Example 28. A system according to any one of the previous examples, wherein a surface of said optics assembly at said optics assembly distal end is curved or is angled relative to a long axis of the cryotherapy device, and wherein said at least one washing opening is position, shaped and sized to release washing fluid towards said optics assembly surface.
   Example 29. A system according to example 28, wherein said optics assembly surface at said optics assembly distal end is substantially perpendicular to said long axis, or is positioned at an angle between 5 degrees to 90 degrees relative to said long axis.
   Example 30. A cryotherapy system, comprising,
an elongated cryotherapy device having an elongated body, a proximal end and a distal end, wherein said device is shaped and sized to be positioned inside a hollow organ, comprising:
a cryo inflow path inside said elongated body fluidically connecting a cryogenic fluid source with said distal end of said cryotherapy device, wherein said cryo inflow path is configured to allow cryogenic flow from said cryogenic fluid source into said hollow organ;
at least one fluid evacuation flow path inside said elongated body having at least one first evacuation opening at a distal end of said at least one fluid evacuation flow path, and at least one second evacuation opening in a circumference of a wall of said evacuation flow path, wherein said first evacuation opening is located distally to said at least one second evacuation opening;
wherein said at least one fluid evacuation flow path is configured to remove fluid and/or particles from said hollow organ through said at least one first evacuation opening and/or through said at least one second evacuation opening;
at least one evacuation seal located within said at least one fluid evacuation flow path between said at least one first evacuation opening and said at least one second evacuation opening, and configured to block at least 10% of evacuation fluid flow through said at least one first evacuation opening.
   Example 31. A system according to example 30, comprising:
an optics assembly having a distal end, positioned at least partly inside said elongated body, wherein said optics assembly is configured to visualize a field of view between said device distal end and a target region inside said hollow organ;
wherein said optics assembly distal end is positioned distally to said at least one second evacuation opening, and wherein said at least one evacuation seal is positioned between said optics assembly distal end and said at least one second evacuation opening.
   Example 32. A system according to example 31, comprising an outer sleeve surrounding said device, and wherein said at least one second evacuation opening is an opening in said outer sleeve or is in a fluid communication with at least one opening in said outer sleeve.
   Example 33. A cryotherapy system, comprising:
an elongated cryotherapy device having an elongated body, a proximal end and a distal end, wherein said device is shaped and sized to be positioned inside a hollow organ, comprising:
a cryo inflow path inside said elongated body fluidically connecting a cryogenic fluid source with said distal end of said cryotherapy device, wherein said cryo inflow path is configured to allow cryogenic flow from said cryogenic fluid source into said hollow organ;
a washing inflow path inside said elongated body comprising a plurality of washing openings at said device distal end axially and/or angularly distributed in a wall of said washing inflow path, wherein said washing inflow path fluidically connects a washing fluid source with said plurality of washing openings, and wherein said washing inflow path is configured to release washing fluid within said hollow organ through at least one washing opening of said plurality of washing openings.
   Example 34. A system according to example 33, wherein said plurality of washing openings are evenly distributed in said wall.
   Example 35. A system according to example 33, wherein said plurality of washing openings are distributed in varying densities of openings per area in said wall.
   Example 36. A system according to any one of examples 33 to 35, wherein said plurality of washing openings have different shapes and/or widths.
   Example 37. A system according to any one of examples 33 to 36, wherein said plurality of openings are divided into 2 or more groups of openings according to shape and/or width of said openings, wherein openings in each 2 or more groups have a similar shape and/or width.
   Example 38. A system according to any one of examples 33 to 37, wherein said plurality of washing openings are located in a portion of said wall of said washing inflow path surrounding and/or along said washing inflow path.
   Example 39. A system according to any one of examples 33 to 37, wherein said plurality of washing openings are located in a portion of said wall of said washing inflow path surrounding at least 10% of a circumference of said inflow path and/or maintaining an axial distance of at least 0.05 mm between two adjacent openings.
   Example 40. A system according to any one of examples 33 to 39, wherein said plurality of washing openings have a maximal width or diameter in a range of 0.01 mm-2 mm.
   Example 41. A system according to any one of examples 33 to 40, comprising:
an optics assembly having a distal end, located inside said elongated body configured to visualize a field of view between said device distal end and a target region inside said hollow organ, and wherein at least some of said plurality of openings are positioned in said wall of said washing inflow path to face said distal end of said optics assembly, and to deliver washing fluid towards said optics assembly distal end.
   Example 42. A system according to any one of examples 33 to 35, wherein said washing inflow path comprises a narrowed portion, and wherein at least some of said plurality of openings are positioned at a wall of said narrowed portion and apply suction force on fluids in said hollow organ.
   Example 43. A cryotherapy system, comprising:
an elongated cryotherapy device having an elongated body, a proximal end and a distal end, wherein said device is shaped and sized to be positioned inside a hollow organ, comprising:
a cryo inflow path inside said elongated body fluidically connecting a cryogenic fluid source with said distal end of said cryotherapy device, wherein said cryo inflow path comprises an opening configured to deliver cryogenic fluid from said cryogenic fluid source into said hollow organ;
wherein at least one surface of said device at said distal end is pretreated to prevent or reduce adhesion and/or formation of one or more liquid drops on the pretreated surface.
   Example 44. A system according to example 43, wherein said device comprises a washing inflow path inside said elongated body comprising at least one washing openings at said device distal end, and configured to release washing fluid within said hollow organ, and wherein said at least one pretreated surface is an external surface of said washing inflow path.
   Example 45. A system according to any one of examples 43 or 44, wherein said device comprises an optics assembly having a distal end inside said elongated body configured to visualize a field of view within said hollow organ between said device distal end and a target region inside said hollow organ; and
wherein said at least one pretreated surface is an external surface of said optics assembly.
   Example 46. A system according to any one of examples 43 to 45, wherein said at least one surface is coated with a hydrophilic or a hydrophobic coating.
   Example 47. A cryotherapy method, comprising:
introducing a cryotherapy device into a hollow organ;
delivering cryofluid into said hollow organ through a cryo fluid inflow path of said cryotherapy device;
releasing washing fluid before, during and/or after said cryofluid delivering into said hollow organ through at least one washing opening of a washing fluid inflow path having a narrowed portion.
   Example 48. The method of example 47, wherein said washing releasing comprises, increasing flow speed of said washing fluid into said hollow organ by said narrowed portion of said washing fluid inflow path.
   Example 49. The method of any one of examples 47 or 48, wherein said releasing comprises releasing said washing fluid into said hollow organ through at least one washing opening located at a wall of said washing fluid inflow path, and proximally to said narrowed portion or in said narrowed portion.
   Example 50. The method of any one of examples 47 to 49, wherein said releasing comprises releasing said washing fluid into said hollow organ through a forward facing distal opening of said washing fluid inflow path.
   Example 51. The method of any one of examples 47 to 50, wherein said introducing comprises introducing said cryotherapy device via a sleeve into said hollow organ.
   Example 52. The method of example 51, comprising:
evacuating fluid from said hollow organ through at least one opening in a circumference of said sleeve.
   Example 53. The method of example 52, comprising:
visualizing a treatment space in said hollow organ into which said cryofluid is delivered by at least one lens or aperture located within said hollow organ, and wherein said evacuating comprises evacuating said fluid through at least one opening in a circumference of said sleeve and proximally to said at least one lens or aperture.
   Example 54. The method of any one of examples 47 to 53, wherein said at least one washing opening comprises a plurality of washing openings having different sizes, and/or widths or diameters, and wherein said releasing comprises releasing said washing fluid through said plurality of openings at different directions within said hollow organ and/or to different distances within said hollow organ according to a shape, size and/or diameter of each opening of said plurality of openings.
   Example 55. The method of any one of examples 47 to 54, wherein said hollow organ comprises a single opening, and wherein said introducing comprises introducing said cryotherapy device into said hollow organ through said single opening.
   Example 56. The method of any one of examples 47 to 55, wherein said hollow organ comprises a bladder, and wherein said introducing comprises introducing said cryotherapy device into said bladder through a urethra.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, microcode, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, such as determine pressure and/or temperature within a body lumen, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1A is a flow chart of a process for applying of washing fluid and controlling the flow of the washing fluid, according to some exemplary embodiments of the invention;

FIG. 1B is a block diagram of a system for delivery of cryotherapy, according to some exemplary embodiments of the invention;

FIG. 2A is a schematic longitudinal cross section view of a cryotherapy device distal end positioned within a body lumen, according to some exemplary embodiments of the invention;

FIG. 2B is a schematic front view of the cryotherapy device distal end of FIG. 2A, according to some exemplary embodiments of the invention;

FIG. 3 is a schematic longitudinal cross section view of a cryotherapy device distal end with liquid drops at different locations of the cryotherapy device, according to some exemplary embodiments of the invention;

FIGS. 4A-4D are schematic longitudinal cross section views of a cryotherapy device distal end, having a plurality of washing fluid openings at different locations of the device, according to some exemplary embodiments of the device;

FIGS. 5A-5B are schematic illustrations of a washing fluid channel having two types of washing fluid openings axially and circumferentially distributed on the external surface of the washing fluid channel, according to some exemplary embodiments of the invention;

FIGS. 5C-5E are schematic illustrations of a washing fluid channel having openings divided into different regions, where openings at one region are partially circumferentially distributed on the external surface of the washing fluid channel, according to some exemplary embodiments of the invention;

FIGS. 6A and 6B are schematic illustrations showing distribution of washing fluid openings when covering an entire surface of a visualization assembly distal end optionally comprising a window or a lens, according to some exemplary embodiments of the invention;

FIGS. 6C and 6D are schematic illustrations showing distribution of washing fluid openings when partially covering a surface of a visualization assembly distal end optionally comprising a window or a lens, according to some exemplary embodiments of the invention;

FIGS. 6E and 6F are schematic cross section views of a cryotherapy device distal end which includes at least one evacuation flow seal, according to some exemplary embodiments of the invention;

FIGS. 7A and 7B are schematic longitudinal cross section views of a cryotherapy device distal end, having flow reducer, for example an internal reducer in a washing fluid channel, according to some exemplary embodiments of the invention;

FIG. 7C is a schematic longitudinal cross section view of a cryotherapy device distal end. having an internal reducer with integrated channels and/or openings, in a washing fluid channel, according to some exemplary embodiments of the invention;

FIG. 7D is a schematic longitudinal cross section view of a cryotherapy device distal end, having an internal reducer in a washing fluid channel and a plurality of separate openings distributed on the surface of the channel, according to some exemplary embodiments of the invention;

FIGS. 8A-8B are schematic cross section views of a cryotherapy distal end having at least one flow reducer located distally to washing openings, for example washing fluid openings within an additional flow reducer, according to some exemplary embodiments of the invention;

FIG. 8C is a schematic cross section front view showing a flow reducer with openings surrounding a distal end cryofluid opening, according to some exemplary embodiments of the invention;

FIGS. 8D and 8E are schematic cross section views showing a distal end of a cryotherapy device comprising a washing fluid flow reducer having a plurality of openings and an evacuation flow seal, according to some exemplary embodiments of the invention;

FIGS. 8F and 8G are schematic cross section views showing a distal end of a cryotherapy device comprising a washing fluid flow reducer, spaced apart washing fluid openings and an evacuation flow seal, according to some exemplary embodiments of the invention;

FIG. 9 is a schematic longitudinal cross section view of a cryotherapy device distal end where at least a region of an external surface of said cryotherapy device exposed to cryo fluid in a body lumen is a non-smooth rough surface, according to some exemplary embodiments of the invention; and

FIGS. 10A-10C are longitudinal cross section views of a cryotherapy device distal end. having a standing, perpendicular surface at the distal end (10A), an angled surface at the distal end (10B), or a curved surface at the distal end (10C), according to some exemplary embodiments of the invention.

### DESCRIPTION OF SPECIFIC EMBODIMENTS OF THE INVENTION

The present invention, in some embodiments thereof, relates to a cryotherapy device and, more particularly, but not exclusively, to a cryotherapy device for treating hollow organ diseases. In some embodiments, the hollow organ diseases comprise bladder cancer, interstitial cystitis, overactive bladder, gastric superficial neoplastic lesions, superficial gastric carcinoma, and/or abdominal wall tumors.

An aspect of some embodiments relates to controlling flow of washing fluid within a body lumen, for example a hollow organ to disrupt existing liquid drops and/or to prevent formation of liquid drops on a surface of a cryotherapy device within the body lumen. In some embodiments, the flow of washing fluid within at least one washing inflow path, for example a washing fluid channel of a cryotherapy device is controlled by at least one flow reducer, for example an internal reducer located within the washing fluid channel. In some embodiments, the at least one flow reducer narrows, for example to at least partly blocks, the washing fluid channel by at least 10%, for example at least 30%, at least 50%, at least 70%, at least 90% or any intermediate, smaller or larger narrowing percentage of the washing fluid channel.

According to some embodiments, the at least one flow reducer within the washing fluid channel controls flow of washing fluid through one or more distal openings of the washing fluid channel, for example forwardly facing openings, optionally facing a target region. In some embodiments, the at least one flow reducer controls at least one of a pressure of washing fluid exiting through the openings, flow speed and/or direction of the washing fluid exiting through the one or more distal openings of the washing fluid channel. Optionally, the at least one flow reducer is located at a distal end of the washing fluid channel, and narrows and/or shapes the one or more distal openings of the washing fluid channel.

According to some embodiments, the at least one flow reducer comprises one or more openings or channels on a circumference of the flow reducer, configured to direct washing fluid to sideways openings on a circumference of the washing channel. In some embodiments, a width of the flow reducer openings determines at least one of an amount of washing fluid directed to the washing channel openings and/or pressure of washing fluid within the flow reducer openings. In some embodiments, an angle of the channels within the flow reducer, for example relative to an axis of the washing fluid channel determines an angle of spraying of the washing fluid outside from the washing channel. In some embodiments, the washing fluid speed flow within the channel relative to the width of the reducer openings controls pressure within the fluid flow channel, for example high speed of washing fluid flow and/or narrow openings may generate negative pressure within the washing fluid channel.

According to some exemplary embodiments, the at least one internal reducer channels and/or openings are shaped, sized and/or aligned to spray washing fluid at a selected deflection angle in a range of 10-170 degrees, for example 10-50 degrees, 30-100 degrees, 90-170 degrees or any intermediate, smaller or larger angle relative to an external surface of the washing fluid channel. Alternatively or additionally, the plurality of internal reducer channels and/or openings are shaped, sized and/or aligned to spray washing fluid at an impingement angle in a range of 0-90 degrees, for example 0-30 degrees, 10-40 degrees, 20-70 degrees, 50-90 degrees or any intermediate, smaller or larger angle or range of angles relative to a surface of the cryotherapy device and/or relative to an optics surface, within the body lumen.

According to some exemplary embodiments, the cryotherapy device comprises at least one flow reducer within a washing fluid channel, located at a distance from openings on the circumference of the washing fluid channel, for example sideways openings optionally facing a surface of a visualization assembly, also termed herein in some embodiments an optics assembly. In some embodiments, a proximal end of the at least one flow reducer is located at a distance of at least 0.5 mm from the openings in the circumference, for example at a distance of at least 1 mm, at least 5 mm, at least 20 mm or any intermediate, smaller or larger distance from at least one opening on the circumference of the washing fluid channel.

An aspect of some embodiments relates to washing fluid channel openings on a circumference of the washing fluid channel with predetermined arrangement, size and distribution, for example to control flow and direction of washing fluid within the body lumen. In some embodiments, a plurality of washing fluid openings are axially and/or circumferentially distributed on a circumference of at least one washing fluid channel within the body lumen. In some embodiments, the plurality of washing fluid openings are optionally shaped, sized, arranged and/or positioned to direct washing fluid towards a surface at the cryotherapy device distal end which is exposed to the formation or attachment of liquid drops, for example to disrupt existing liquid drops already attached to the surface and/or to prevent attachment or formation of the liquid drops on the surface. Alternatively or additionally, the plurality of washing fluid openings are optionally shaped, sized, arranged and/or positioned to direct washing fluid towards a lumen of at least one evacuation flow path, for example to clear the flow path and prevent blockage of the evacuation flow path by liquid drops. Alternatively or additionally, the plurality of washing fluid openings are optionally shaped, sized, arranged and/or positioned to direct washing fluid towards a space between the visualization assembly and a treatment region, for example to generate a flow protecting space.

According to some embodiments, the plurality of openings optionally have a similar shape and/or size. Alternatively, at least some of the openings optionally have a different shape and/or size. In some embodiments, the plurality of openings have a size, for example a maximal width or a maximal diameter in a range of 0.01 mm-2 mm, for example 0.05 mm-0.5 mm, 0.07 mm-1 mm, 0.8 mm-2 mm or any intermediate, smaller or larger range of values.

According to some embodiments, the plurality of openings are axially and/or circumferentially distributed on the outer surface of the washing fluid channel, for example in at least one region of the wall facing at least one selected location, for example at least one region of the wall facing a field of view (FOV) between a visualization assembly and a treated tissue, at least one region of the wall facing the visualization assembly and/or at least one region of the wall facing a lumen or an opening of an evacuation flow path. In some embodiments, the plurality of openings are circumferentially distributed along the circumference of the washing fluid channel. Alternatively or additionally, at least some of the plurality of openings are distributed along a portion of the circumference, for example along an arc subtending an angle smaller than 360 degrees, smaller than 180 degrees, smaller than 90 degrees or any intermediate, smaller or larger value. Alternatively or additionally, at least some of the plurality of openings are distributed along part of a surface connected or adjacent to said washing fluid channel.

According to some embodiments, the plurality of washing openings are located in a portion of a wall of the washing channel, for example a washing inflow path surrounding and/or along the washing inflow path. Alternatively or additionally, the plurality of washing openings are located in a portion of the wall of the washing inflow path surrounding at least 5%, for example at least 10%, at least 20%, at least 30% or any intermediate, smaller or larger range of values, of a circumference of the inflow path and/or maintaining an axial distance of at least 0.05 mm, for example at least 0.2 mm, at least 1 mm or any intermediate, smaller or larger value, between two adjacent openings.

According to some embodiments, at least some of the plurality of openings have different shapes and/or sizes. In some embodiments, at least some of the plurality of openings are arranged in an array of openings. In some embodiments, the plurality of openings is distributed evenly in the array. Alternatively, a distance between at least some adjacent openings in the array, varies.

Potential advantages of controlling flow of washing fluid may be to allow efficient removal of liquid drops that interfere with visualization and may promote or transfer cryo frost to sensitive locations, for example, liquid drops on optic lens, liquid drops on tubes, and liquid drops inside evacuation path.

According to some embodiments, cryotherapy devices are designed with different shapes and/or sizes of washing fluid openings, for example according to a clinical application, according to the hollow organ, and/or according to humidity levels in the hollow organ. For example, in some embodiments, if the wash pressure is low and the outer ambient pressure in the body lumen is high, the washing flow openings are designed to be very small, for example in a range of 0.01 mm-0.3 mm, 0.01-0.1 mm, 0.01 mm-0.5 mm, 0.5-0.2 mm or any intermediate, smaller or larger range of values, optionally to keep liquids from entering from the body lumen into the washing channel. Alternatively, if the wash pressure is high and/or outer ambient pressure in the body lumen is low, a size of the washing fluid openings is in a range of 0.5-2 mm, for example 0.5 mm-1 mm, 0.7 mm-1.5 mm, 1 mm-2 mm or any intermediate, smaller or larger range of values. In some embodiments, a cryotherapy device with a specific arrangement of washing flow openings is selected according to a specific clinical application, and/or a specific hollow organ.

An aspect of some embodiments relates to directing evacuation of fluid and particles from a treatment space within a body lumen, around a visualization assembly of the cryotherapy device. In some embodiments, fluid flow evacuation from the treatment space and/or the body lumen is directed towards one or more evacuation openings located proximally to a distal end of the visualization assembly. In some embodiments, the one or more evacuation openings are openings in an outer sleeve of the cryotherapy device located within the body lumen. In some embodiments, the one or more evacuation openings are openings of at least one evacuation channel passing through an inner lumen of the sleeve, or are directed towards openings of at least one evacuation channel.

According to some embodiments, the cryotherapy device comprises at least one evacuation seal, for example a distal evacuation seal configured to at least partly block evacuation of fluid and particles through a distal, for example forwardly facing opening of the evacuation channel. In some embodiments, the forwardly facing opening is an opening of the evacuation channel facing a treatment space and/or a target of a cryotherapy treatment within the body lumen. In some embodiments, the at least one distal evacuation seal reduces the passage of fluid and particles through the forwardly facing opening of the evacuation channel by at least 50%, for example by at least 60%, by at least 70%, by at least 80%, by at least 90% or any intermediate, smaller or larger percentage value.

According to some exemplary embodiments, the distal evacuation seal is axially located on the cryotherapy device proximally to the visualization assembly distal end. In some embodiments, the distal evacuation seal is located between a distal end of the visualization assembly and the evacuation openings in the outer sleeve.

In some embodiments, blocking a passage of fluid and particles by the evacuation seal, directs the fluid and particles around the visualization assembly towards the evacuation openings in the outer sleeve.

According to some embodiments, the distal evacuation seal is an adjustable seal. In some embodiments, a size of at least one opening in the seal can be adjusted, for example to increase or reduce flow of fluid and/or particles into the at least on evacuation channel. In some embodiments, the seal opening size is adjusted according to signals received from at least one sensor, optionally indicating increase of pressure within the body lumen and/or clogging of the evacuation openings in the outer sleeve.

An aspect of some embodiments relates to modifying or pretreating a surface of the cryotherapy device in order to prevent or reduce attachment and/or formation of liquid drops on the modified surface. In some embodiments, at least a portion of the surface is modified into a non-smooth rough surface. In some embodiments, the non-smooth rough surface changes, for example reduces an adhesion force, or a behavior of the adhesion force, between a liquid drop and the surface.

According to some exemplary embodiments, the pretreated surface is optionally formed during manufacturing, for example by coating a surface with hydrophilic or hydrophobic coatings. Alternatively, a non-smooth rough surface is formed for example by sandblasting, electro etching or other surface thermal treatments.

An aspect of some embodiments relates to evacuating fluids from a hollow organ through at least one evacuation side opening. In some embodiments, the at least one evacuation side opening is located in a circumference of a cryotherapy device body, for example a sleeve. In some embodiments, the at least one evacuation opening comprise a plurality of evacuation openings. In some embodiments, the plurality of evacuation openings are axially and/or angularly distributed in a wall of said cryotherapy body, for example in a wall of the sleeve.

According to some embodiments, at least some of the plurality of openings are located proximally to a distal end of a visualization assembly optionally comprises a lens, an aperture and/or illumination.

Potential advantages of treating a surface may be to prevent or reduce accumulation or formation of liquid drops, to reduce drops profile for example direct liquids to form one or more shallow layers instead of drops, in locations where there is limited access for washing fluid or where washing fluid flow is not strong enough to dry or break the liquid drops.

According to some embodiments, the methods, system and/or devices describe herein are used for treating bladder cancer, for example by delivery of cryofluid towards malignant tissue, for example cancerous tissue, in the bladder.

According to some embodiments, the cryotherapy device comprises an elongated body, optionally a tubular body. In some embodiments, at least one washing fluid channel passes within the elongated body, optionally surrounding at least partly a cryofluid channel of the device. In some embodiments, washing fluid is released from at least one opening of the washing channel, optionally outwardly towards at least one portion of a visualization assembly located between the opening of the washing channel and an external surface of the device body.

According to some embodiments, controlling the release of washing fluid towards a visualization assembly and/or towards a FOV, as described herein, is performed in a device that does not include a cryofluid channel, for example a device that includes a visualization assembly and a washing fluid channel. In some embodiments, the device includes at least one of a flow reducer, a plurality of openings or channels for controlling the release of the washing fluid towards the visualization assembly. A potential advantage of including a cryofluid channel is that it may allow to reduce the number of devices inserted into a hollow organ.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Exemplary General Process for Applying Washing Fluid

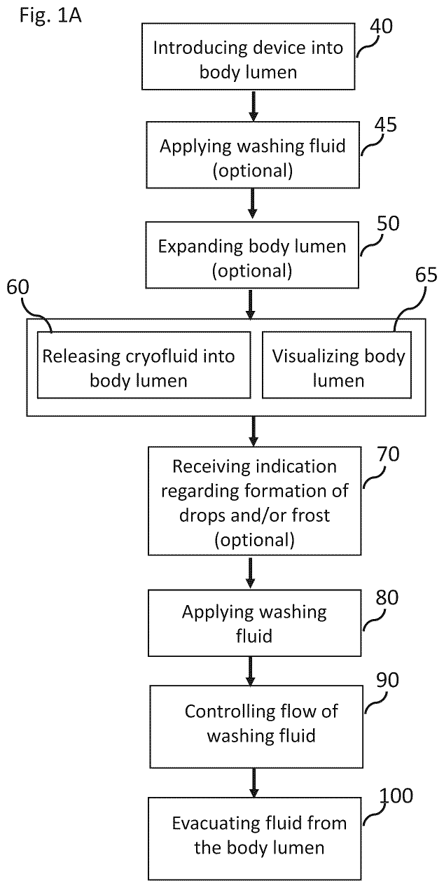

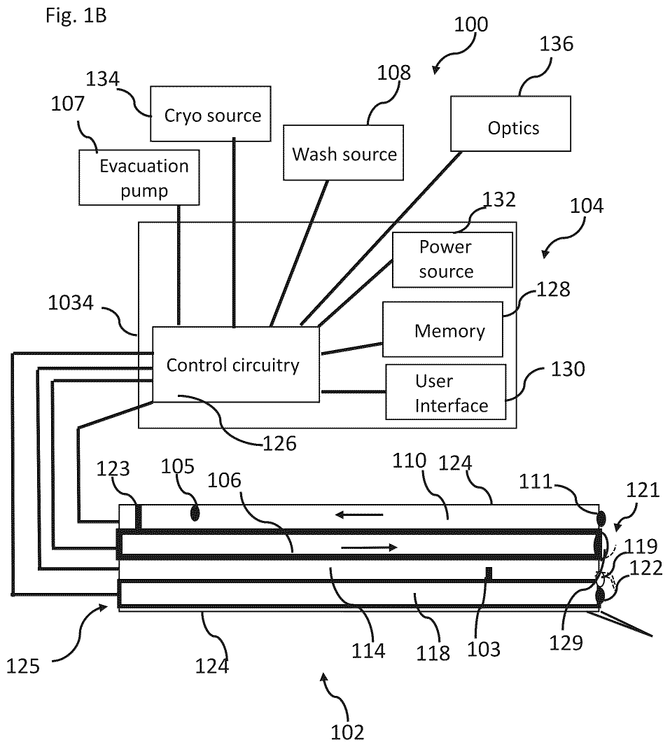

According to some exemplary embodiments, a cryo-ablation treatment, for example cryotherapy, is delivered within a body lumen, for example a hollow organ, by a cryotherapy device. In some embodiments, the body lumen is a body lumen with a single opening, which is optionally narrow. In some embodiments, the body lumen optionally comprises body fluids and/or humidity. In some embodiments, release of cryo fluid within the body lumen, may cause a formation of liquid drops and/or frost within the body lumen, on at least one surface of a cryotherapy device, within at least one internal lumen of the cryotherapy device and/or on a surface of an additional device inserted into the body lumen. In some embodiments, in order to at least one of prevent, reduce, and remove the liquid drops and/or frost, washing fluid is applied. Reference is now made to FIG. 1A, depicting a process for application of washing fluid within a body lumen, for example a hollow organ, according to some exemplary embodiments of the invention.

According to some exemplary embodiments, at least part of a cryotherapy device is introduced into a body lumen, at block 40. In some embodiments, a distal end of the cryotherapy device is optionally introduced into the body lumen. In some embodiments, the distal end of the cryotherapy device comprises at least one cryo fluid release opening, configured to introduce cryo fluid into the body lumen, and at least one evacuation opening, configured to remove fluid from the body lumen. In some embodiments, a distal end of the cryotherapy device optionally includes at least one washing fluid opening for delivery of washing fluid into the body lumen. In some embodiments, the distal end of the cryotherapy device additionally comprises a visualization assembly, for example at least one of a lens, optic sensor, camera, an optic fiber end or an optic fibers bundle end configured to allow visualization of the body lumen, for example the inner surface of the body lumen. In some embodiments, the visualization means are configured to allow visualization of the distal end and/or a therapeutic space between the distal end of the cryotherapy device and the inner surface of the body lumen.

According to some exemplary embodiments, the body lumen, for example the hollow organ comprises bladder, renal pelvis, uterus, stomach or abdomen. In some embodiments, the cryotherapy device is introduced into the body lumen at block 40 through a body opening, for example an anatomical opening of the body lumen, or an invasive port, for example a minimal invasive port. In some embodiments, the minimal invasive port comprises a laparoscopic port, for example a laparoscopic port. In some embodiments, the body lumen comprises the bladder, and the cryotherapy device is optionally introduced into the bladder at block 40 through the urethra. Alternatively, the device is optionally introduced into the abdomen through laparoscopic trocar, through the vagina to the uterus, through the esophagus to the stomach etc.

According to some exemplary embodiments, washing fluid is optionally applied, for example released into the body lumen at block 45. In some embodiments, washing fluid is applied, for example to clear a visualization field, or to expand narrow opening when navigating the device into the body lumen and/or within the body lumen. In some embodiments, washing fluid is optionally applied, for example to allow clear visualization of an inner surface of the body lumen, and/or a target region in the inner surface.

According to some exemplary embodiments, washing fluid is optionally released into the body lumen at block 45 through at least one washing opening located in a circumference of a washing fluid inflow path. In some embodiments, the washing fluid inflow path includes a narrowed portion in which flow speed is increased. In some embodiments, the washing fluid is optionally released into the body lumen through at least one washing fluid opening located proximally to the narrowed portion. Alternatively or additionally, washing fluid is optionally released into the body lumen through at least one washing fluid opening located in said narrowed portion. Alternatively or additionally, the washing fluid is optionally released into the body lumen through at least one washing fluid opening located distally to the narrowed portion.

According to some exemplary embodiments, the body lumen is optionally expanded at block 50. In some embodiments, the body lumen is expanded before the introduction of the cryotherapy device into the body lumen. Alternatively, the body lumen is expanded by the cryotherapy device. In some embodiments, the body lumen is expanded, for example to allow better visualization of the inner surface of the body lumen. Alternatively or additionally, the body lumen is expanded in order to flatten the inner surface of the body lumen, for example to allow better access of the cryo fluid released in the body lumen to a target region in the body lumen, for example to a target region on the inner surface of the body lumen.

According to some exemplary embodiments, the body lumen is optionally expanded at block 50 by releasing expandable cryo fluid into the body lumen through at least one opening of the cryotherapy device. Alternatively or additionally, washing fluid is optionally released into the body lumen at block 50 from at least one opening of the cryotherapy device, to expand the body lumen.

According to some exemplary embodiments, cryo fluid is released into the body lumen at block 60. In some embodiments, the cryo fluid is optionally released through at least one opening, for example a nozzle at the distal end of the cryotherapy device. In some embodiments, the released cryo fluid expands and lowers the temperature inside the body lumen, for example at a defined target tissue within the body lumen, for example to ablate a tissue in the target region. Optionally, the cryo fluid is directed towards the target region or towards a treatment space between a distal end of the cryotherapy device and the target region.

According to some exemplary embodiments, the body lumen is visualized at block 65. In some embodiments, the body lumen is visualized in a timed relationship to the releasing of the cryo fluid at block 60, for example before, during and/or after the release of the cryo fluid. In some embodiments, at least one of the target region or the treatment space in the body lumen is visualized at block 65. In some embodiments, the body lumen is visualized by a visualization assembly, for example an optical assembly located at the distal end of the cryotherapy device.

According to some exemplary embodiments, an indication is optionally received regarding a formation of liquid drops and/or frost in the body lumen, at block 70. In some embodiments, the indication is a visual indication received from the visualization assembly. Alternatively, the indication is received from at least one sensor of the cryotherapy device, for example from at least one sensor located at the distal end of the cryotherapy device and/or along the cryotherapy device. In some embodiments, the at least one sensor comprises a temperature sensor, a flow sensor, a humidity sensor and/or an image sensor. In some embodiments, a control unit, for example a control circuitry of the control unit, coupled to the cryotherapy device receives the indication based on signals received from the visualization assembly and/or the at least one sensor.

According to some exemplary embodiments, washing fluid is applied, at block 80. In some embodiments, the washing fluid is applied in response to the indication optionally received at block 70. Alternatively, application of the washing fluid is a preventive process, for example washing fluid is applied without receiving an indication at block 70. In some embodiments, washing fluid is applied intermittently, optionally a short bursts. In some embodiments, the washing fluid is applied intermittently, for example every 0.1 seconds, every 0.5 seconds, every 2 seconds or any intermediate, smaller or larger time period. In some embodiments, washing fluid, for example washing liquid or washing gas is applied into the body lumen. In some embodiments, the washing fluid is applied towards at least one of, at least part of the visualization assembly, the treatment target, the treatment space, at least one surface of the cryotherapy device located in the body lumen. In some embodiments, the washing fluid is applied to prevent the formation of liquid drops and/or frost in the body lumen. Alternatively or additionally, the washing fluid is optionally applied in order to prevent attachment and/or accumulation of the liquid droplets in the body lumen, on a surface or opening of the cryotherapy device and/or on the visualization assembly. Alternatively or additionally, the washing fluid is optionally applied in order to prevent attachment and/or accumulation of the frost in the body lumen, on a surface or opening of the cryotherapy device and/or on the visualization assembly. Alternatively or additionally, the washing fluid is optionally applied in order to clear a field of view (FOV) of the visualization assembly.

According to some exemplary embodiments, the washing fluid is applied at block 80 through at least one opening of the cryotherapy device, for example at least one opening at the distal end of the cryotherapy device. In some embodiments, the at least one opening comprises a plurality of openings. In some embodiments, the plurality of openings is located at 2 or more locations at the distal end of the cryotherapy device.

According to some exemplary embodiments, washing fluid is released into the body lumen at block 80 through at least one washing opening located in a circumference of a washing fluid inflow path. In some embodiments, the washing fluid inflow path includes a narrowed portion in which washing fluid flow speed is increased. In some embodiments, the washing fluid is optionally released into the body lumen through at least one washing fluid opening located proximally to the narrowed portion. Alternatively or additionally, washing fluid is optionally released into the body lumen through at least one washing fluid opening located in said narrowed portion. Alternatively or additionally, the washing fluid is optionally released into the body lumen through at least one washing fluid opening located distally to the narrowed portion.

According to some exemplary embodiments, flow of the washing fluid is controlled, at block 90. In some embodiments, the flow of the washing fluid is controlled by applying the washing fluid through openings having a predetermined size, a predetermined shape and/or a predetermined location. In some embodiments, the size and/or the shape of all washing fluid openings is identical. Alternatively, at least some of the washing fluid openings has a different shape and/or size. In some embodiments, the size, shape and/or location of the openings allows to control the distribution and/or direction of the washing fluid within the body lumen. Alternatively or additionally, the size, shape and/or location of the openings determine the distribution and/or direction of the washing fluid with respect to at least one outer surface of the cryotherapy device and/or the visualization assembly.

According to some exemplary embodiments, the flow of washing fluid is controlled at block 90, for example by controlling the amount of washing fluid directed within the cryotherapy device towards the openings. In some embodiments, the amount of washing fluid directed towards the openings is controlled, for example by controlling a width of the washing fluid flow path within the cryotherapy device. Alternatively or additionally, the amount of washing fluid directed towards the openings is controlled, for example by controlling or restricting an internal cross section of a channel in which washing fluid flows. Alternatively or additionally the amount of washing fluid directed towards the openings is controlled, for example by opening at least valve, for example a valve in a washing fluid channel.

According to some exemplary embodiments, fluid is evacuated from the body lumen at block 100. In some embodiments, fluid is evacuated from the body lumen, for example in order to maintain a target, for example a predetermined, pressure level and/or temperature level within the body lumen. Alternatively or additionally, fluid is evacuated from the body lumen, for example to allow better visualization at block 65. Alternatively or additionally in some embodiments, fluid is evacuated from the body lumen before, during and/or after the application of washing fluid at blocks 45 and/or 80. Alternatively or additionally, fluid is evacuated from the body lumen optionally automatically, in response to a signal received from at least one temperature and/or at least one pressure sensor.

According to some exemplary embodiments, fluid is evacuated through at least one evacuation side opening in an elongated hollow body, for example a sleeve or overtube used during the introducing of the cryotherapy device, at block 40. In some embodiments, the at least one evacuation side opening is an opening in a circumference of the sleeve or overtube. In some embodiments, the at least one evacuation side opening is positioned within the body lumen. Alternatively or additionally, fluid is evacuated through at least one distal opening of at least one evacuation flow path of said cryotherapy device, positioned within the body lumen.

### Exemplary Cryotherapy System

According to some exemplary embodiments, a cryotherapy system comprises a cryotherapy probe configured to be inserted at least partly into a body lumen, and a control unit connected to the cryotherapy device. In some embodiments, the control unit is positioned outside the body. Reference is now made to FIG. 1B depicting a cryotherapy system, according to some exemplary embodiments of the invention.

According to some exemplary embodiments, cryotherapy system 100 comprises a cryotherapy probe, for example cryotherapy device 102, and a control unit 104 connected to the cryotherapy device 102. In some embodiments, the cryotherapy device 102, for example a body of the cryotherapy device, is an elongated probe having a distal end 121, optionally shaped and sized to be introducible into a body lumen and to face a target region in the internal surface of the lumen, and a proximal end 125, optionally positioned outside the body lumen. Optionally, the cryotherapy device 102, for example a body of the cryotherapy device is hollow, for example to allow positioning of one or more flow paths, one or more channels and/or assemblies, for example an optics assembly within the device body. In some embodiments, the cryotherapy device 102 is cylindrical, and has a diameter at the distal end 121 of the cryotherapy device and the sleeve around it in a range of 3 to 15 mm, for example 3 mm, 5 mm, 7 mm, 9 mm or any intermediate, smaller or larger value. In some embodiments, the diameter of a cryotherapy device not including a sleeve, is in a range of 0.3 to 5 mm, for example in a range of 0.3-0.8 mm, 0.5-1.5 mm, 0.8-2 mm 0.5 mm, or any intermediate, smaller or larger value. In some embodiments, the cryotherapy device 102 is shaped and sized to be introduced into the body lumen through a working channel of an endoscope. Alternatively, the cryotherapy device 102 is introduced through a hollow elongated body of the cryotherapy system, for example sleeve 124 into the body lumen. Alternatively, outer shape of the cryotherapy device 102 is built/has dedicated structure, to be introduced into the body lumen (e.g. a dedicated scope+cryo clement).

Alternatively, the cryotherapy system comprises an endoscope, optionally including the visualization assembly, and a cryotherapy device not including a visualization assembly that is introduced through a working channel of the endoscope.

According to some exemplary embodiments, the cryotherapy device 102 comprises at least one flow path, for example a lumen or a channel passing through a lumen, of the cryotherapy, device from the proximal end 125 to the distal end 121. In some embodiments, the at least one flow path is used as an inflow path towards the distal end 121 and into the body lumen, and as an outflow path from the body lumen towards the proximal end 125. In some embodiments, the cryotherapy device 102 comprises at least one inflow path, for example inflow channel 106 for delivering fluid into the body lumen. Additionally or optionally, the cryotherapy device 102 comprises at least one outflow path, for example outflow channel 110 for evacuating fluids, liquids, particles and/or gas from the body lumen.

According to some exemplary embodiments, the outflow path, for example outflow channel 110 comprises at least one outflow flow regulator, for example valve 123 for controlling the passage of material, for example fluids or particles through the outflow path. In some embodiments, the at least one valve 123 comprises at least one check valve, configured to be opened, optionally passively, when a pressure inside the body lumen exceeds a pre-determined value. Optionally, the outflow flow regulator is positioned near a proximal end 125 of the cryotherapy device 102, located outside the body.

According to some exemplary embodiments, the inflow channel 106 comprises at least one cryo-nozzle 129, for example a forward facing cryo-nozzle and/or a sideways facing cryo-nozzle configured to spray cryogenic fluid, for example cryogenic gas and/or cryogenic liquid towards a selected target region on the internal surface of the lumen. Optionally, the cryogenic fluid is stored in high-pressure. In some embodiments, and without being bound by any theory, the expansion of the high-pressure cryogenic fluid within the body lumen, lowers the pressure of the fluid rapidly which results with a cryogenic effect inside the body lumen, for example the Joule-Thomson effect.

According to some exemplary embodiments, the cryo-nozzle 129 is an adjustable cryo-nozzle configured to spray the cryogenic fluid at an angle smaller or larger than 90 degrees to the target region. Alternatively, the cryo-nozzle 129 is a fixed angle cryo-nozzle, fixed at an angle different than 90 degrees, for example 15 degrees, 30 degrees, 45 degrees, 55 degrees or any intermediate, smaller or larger angle different than 90 degrees. Alternatively, the angle is 90 degrees. In some embodiments, the adjustable cryo-nozzle is configured to control the amount of the sprayed cryogenic fluid released through the nozzle to the lumen, for example by adjusting the opening diameter of the cryo-nozzle. Alternatively or additionally, the amount of the sprayed cryogenic fluid released through the nozzle to the lumen is controlled by opening at least one flow valve.

According to some exemplary embodiments, the cryotherapy device 102 comprises at least one optical assembly, for example electrical wires, optic fibers, optical prisms, optics path, optionally optics channel 118, for delivering images and/or visual signals of the tissue facing the distal end 121 of the cryotherapy device 102, to an optic sensor located outside the body, for example an optic sensor in the control unit 104, or to an external optical assembly, for example an optical assembly of a different control unit. Alternatively, the optical assembly comprises at least one optic sensor, for example optic sensor 122 at the distal end 121 of the cryotherapy device 102, configured to sense visual signals of the tissue facing the distal end 121 of the cryotherapy device 102, for example images and/or visual signals of a target region within the body lumen. Alternatively, the optical assembly comprises a sensor optionally an Ultra-Sound (US) sensor or a magnetic resonance (MR) sensor.

According to some exemplary embodiments, the cryotherapy device 102 comprises at least one illumination source, for example illumination source 119. In some embodiments, the illumination source 119 is positioned at the distal end 121 of the cryotherapy device, optionally facing the tissue of the body lumen. Alternatively, the at least one light source is positioned inside a channel within the cryotherapy device, for example optics channel 118.

According to some exemplary embodiments, the optics channel 118 is or comprises at least one of, an optical fiber, a group of optical fibers or an optical fiber cable. In some embodiments, when the optics channel 118 is or comprises at least one of, an optical fiber, a group of optical fibers or an optical cable, the light source is positioned outside the body, and optionally the optical sensor is positioned outside the body. Alternatively or optionally, the optics channel 118 comprises at least one LED light, optionally connected by wires to an external unit.

According to some exemplary embodiments, the cryotherapy device 102 comprises at least one flow path, for example washing channel 114 for insertion of a washing fluid, for example gas or liquid into the lumen, for example the body lumen. In some embodiments, insertion of the washing fluid, for example the washing gas is used to inflate the body lumen, for example to allow better visualization of the internal surface of the lumen and/or to allow better access to desired regions, optionally regions selected for cryotherapy, within the body lumen. Additionally or alternatively, the washing fluid is sprayed through a washing opening, optionally comprising a nozzle, into the treatment region, for example to clear or to blow away condensed particles and/or clouds of condensed particles. Optionally, the condensed particles and/or the clouds of condensed particles are formed by the interaction of the cryogenic gas with the humidified environment inside the body lumen.

According to some exemplary embodiments, the washing channel 114 comprises at least one washing opening, for example 2, 3, 4, 5, 6, 10, 100 or any larger or intermediate number of washing openings. In some embodiments, the washing channel 114 comprises a plurality of washing openings. In some embodiments, the plurality of washing openings or at least some of the washing openings optionally have the same width or diameter. In some embodiments, the at least some of the washing openings are optionally sideways facing openings. Alternatively, at least some of the washing openings are forward facing openings.

According to some exemplary embodiments, the washing openings are axially and/or angularly distributed in a wall of the washing channel, for example in a wall of a washing inflow path.

According to some exemplary embodiments, at least one, or at least some of the washing openings comprise at least one washing opening at the distal end of the cryotherapy device, for example a washing opening facing the tissue. In some embodiments, the at least one washing opening is optionally a forward facing washing opening. In some embodiments, the at least one washing opening is configured to spray fluid, for example gas, for example low-pressure gas into a space between the cryotherapy device 102 and the target region. In some embodiments, the at least one washing opening is an adjustable opening, for example to allow spraying of washing fluid, for example gas or liquid, sideways or in an angle to the target region, for example a predetermined angle relative to an external surface of the washing fluid channel or relative to a surface of the target region. In some embodiments, the at least one opening sprays washing fluid, for example gas or liquid into a field of view (FOV) of the optic sensor 122 or into the FOV of the optics channel 118.

According to some exemplary embodiments, the cryotherapy device 102 comprises at least one washing director at the washing opening, for example a deflecting surface configured to direct washing fluid towards the optical assembly, for example a lens and/or into FOV of the optic sensor 122 or into the FOV of the optics channel 118. Optionally, the at least one washing director is an adjustable washing director, for example configured to allow spraying wash gas in an angle to the FOV.

According to some exemplary embodiments, at least one washing flow regulator on the washing inflow path is configured to adjust the amount of the released washing fluid through the washing opening, optionally in a response to a signal received from a control unit, and/or in a predefined sequence.

According to some exemplary embodiments, the control unit 104 comprises at least one control circuitry, for example control circuitry 126. In some embodiments, the control circuitry 126 controls the flow of cryogenic fluid through the inflow path, for example inflow channel 106. In some embodiments, the control circuitry 126 controls the flow of the cryogenic fluid by controlling at least one cryo flow regulator, for example a valve positioned inside the inflow path and/or at least one valve between a cryo source 134 and the inflow channel 106. Alternatively or additionally, the control unit 104 controls at least one valve of the cryo-source 134, for example an outlet valve of the cryo-source 134.

According to some exemplary embodiments, the control circuitry 126 controls the flow of a washing fluid through a wash inflow path, for example washing channel 114. In some embodiments, the control circuitry 126 controls the flow of the washing fluid by controlling at least one washing flow regulator, for example a valve positioned on the wash inflow path, for example washing channel 114. Alternatively or additionally, the control circuitry 126 controls the flow of the washing fluid by controlling at least one flow regulator between a wash source 108 and the washing channel 114, for example at least one valve. Alternatively or additionally, the control circuitry 126 controls at least one valve of the wash-source, for example an outlet valve of the wash source 108. In some embodiments, the wash source 108 comprises a pump, for example a pump configured to compress air. Alternatively, the wash source 108 comprises a container of washing fluid.

According to some exemplary embodiments, the washing channel 114 comprises at least one flow reducer 103 in an inner lumen of the washing channel 114. In some embodiments, the flow reducer 103 is shaped and sized to narrow the washing channel 114. In some embodiments, the flow reducer contacts a wall of the washing channel and/or a wall of the cryo fluid inflow channel 106. In some embodiments, the flow reducer 103 blocks at least 10%, for example at least 20%, at least 30%, at least 50%, at least 60%, at least 70% or any intermediate, smaller or larger percentage value of washing fluid flow within the washing channel 114.

According to some exemplary embodiments, the flow reducer is shaped as an arc contacting a wall of the washing channel and/or a wall of the cryo fluid inflow channel 106. Optionally, the flow reducer 103 is shaped as a tube coaxially positioned within the washing channel 114. In some embodiments, the flow reducer 103 comprises a protrusion, protruding into the washing channel 114 from the washing channel wall and/or from a wall of the cryo fluid inflow channel 106, which optionally coaxially positioned within the washing fluid channel 114.

According to some exemplary embodiments, the flow reducer 103 comprises at least one, for example a plurality of channels, optionally located in a circumference of the flow reducer, configured to direct washing fluid into the body lumen. In some embodiments, the at least one flow reducer channel in is aligned with at least one side opening in a wall of the washing channel 114. Optionally, the at least one flow reducer channel is formed from two or more openings in the flow reducer wall, for example in a circumference of the flow reducer.

According to some exemplary embodiments, the control circuitry 126 controls the flow of fluid, for example gas and/or liquid out from the body lumen through the outflow path, for example outflow channel 110. In some embodiments, the control circuitry controls the flow through outflow channel 110 by controlling an outflow flow regulator, for example an outflow valve inside or near a proximal end of the outflow channel, for example valve 123. Alternatively or additionally, the control circuitry 126 controls the activation of an evacuation pump 107, for example a vacuum pump connected to the outflow channel. In some embodiments, the evacuation pump 107 is used to actively evacuate fluid, for example liquid and/or gas and/or particles out from the body lumen through the outflow path of the cryotherapy device. Optionally, the control circuitry 126 controls at least one evacuation flow regulator, for example an evacuation valve on a tube connecting the evacuation pump 107 and the outflow channel. Alternatively, evacuation is performed via a check valve.

According to some exemplary embodiments, the control circuitry 126 controls an optical assembly, for example an optic sensor positioned on the cryotherapy device 102, for example optic sensor 122. Alternatively or additionally, the control circuitry 126 controls at least one optic sensor in the control unit 104. In some embodiments, the control circuitry 126 controls the opening or closing of an optics channel 118, optionally by controlling an aperture within the optics channel 118. In some embodiments, the control circuitry controls the activation of optics 110, for example visualization means.

According to some exemplary embodiments, the cryotherapy device 102 comprises at least one sensor 111 located at the distal end 121, for example to sense at least one environmental parameter of the body lumen. In some embodiments, the environmental parameter comprises temperature, pressure, and/or humidity level or any other environmental parameter. Alternatively or additionally, the cryotherapy device 102 comprises at least one sensor positioned at least partly within the outflow path, for example sensor 105, for sensing the environmental parameter, for example outflow amount, outflow temperature, and/or outflow humidity.

According to some exemplary embodiments, sensor 111 and/or sensor 105 are electrically connected to the control circuitry 126. Alternatively or additionally, the control circuitry 126 is electrically connected to one or more sensors positioned inside or outside the body. In some embodiments, the one or more sensors are positioned near or at a distance from the cryo ablation target region. In some embodiments, at least one of the sensors transmits the measured environmental parameter values to the control circuitry 126. In some embodiments, the control circuitry stores the measured values in memory 128. In some embodiments, the memory 128 comprises predetermined values of at least one environmental parameter, for example maximal pressure values, minimal temperature values or any other predetermined environmental parameter value or indications thereof. In some embodiments, memory 128 comprises at least one cryotherapy protocol, values of at least one cryotherapy parameter or indications thereof. In some embodiments, the memory comprises log files related to the operation of the cryotherapy device 102 or the operation of the cryotherapy system 100.

According to some exemplary embodiments, the control unit 104 comprises at least one user interface 130 functionally connected , for example electrically, optically or wirelessly connected to the control circuitry 126. In some embodiments, the user interface 130 comprises a sound source and/or a display. In some embodiments, the control circuitry 126 signals the user interface 130 to generate at least one indication, for example a human detectable indication to a user of the cryotherapy device or the cryotherapy system. In some embodiments, the control circuitry 126 signals the user interface 130 to generate an alert, for example when the measured environmental parameter values inside the body lumen exceed a pre-determined value.

According to some exemplary embodiments, the control circuitry 126 is functionally connected to at least one cryo flow regulator on the cryo inflow path, for example a valve. In some embodiments, the control circuitry 126 regulates the flow through the cryo inflow path, for example reducing flow within the inflow path, for example stopping flow within the inflow path and/or increasing flow within the inflow path for example by delivering a signal to the cryo flow regulator. In some embodiments, the control circuitry 126 regulates the cryogenic fluid flow within the cryo inflow path, for example when the pressure within the body lumen is higher than a pre-determined pressure value. Alternatively, the control circuitry 126 regulates the cryogenic fluid flow within the cryo inflow path, for example when the temperature inside the body lumen is below a pre-determined temperature value or when receiving an input signal from a user of the system.

According to some exemplary embodiments, the control circuitry 126 controls at least one purge flow regulator on the cryo inflow path. In some embodiments, the purge flow regulator is configured to control flow of low-pressure fluid through the cryo inflow path and optionally through the cryo-nozzle into the body lumen, for example when the cryogenic fluid flow is stopped, reduced or regulated by the control circuitry.

According to some exemplary embodiments, the control circuitry 126 is connected to the outflow flow regulator, for example a valve. In some embodiments, the control circuitry regulates the outflow flow regulator, for example by opening the outflow path, for example when the pressure within the body lumen is higher than a predetermined value and/or when the temperature is lower than a predetermined temperature value.

According to some exemplary embodiments, the control unit 104 comprises a power source, for example power source 132. Alternatively, the control unit 104 is connected to an external power supply for example by electrical wiring, optically, or wirelessly. In some embodiments, the power source comprises a battery, optionally a rechargeable battery.

According to some exemplary embodiments, a control unit, for example control unit 104 is connected to at least one inflow path of a cryotherapy device, configured to allow fluid flow from a fluid flow source into a body lumen. Optionally, the control unit 104 is connected to at least one inflow flow regulator on the inflow path, configured to regulate fluid flow through the inflow path and into the body lumen.

In some embodiments, the control unit, for example control unit 104 is connected to at least one outflow path of a cryotherapy device, configured to allow evacuation of fluid out from the body lumen. Optionally, the control unit 104 is connected to at least one outflow regulator, for example a valve or a check valve on the outflow path, configured to regulate the evacuation of fluids from the body lumen through the outflow path.

In some embodiments, the control unit 104 controls the inflow regulator and the outflow regulator to regulate pressure levels within the body lumen to reach a pressure level lower than a pre-determined value. Alternatively or additionally, the control unit 104 controls the inflow regulator and the outflow regulator to regulate temperature levels within the body lumen to a temperature level higher than a pre-determined temperature value.

According to some exemplary embodiments, the wash source 108 comprises a fluid container, for example a gas container or a liquid container. Alternatively, the wash source 108 comprises a pump configured to pressurize air. In some embodiments, the cryo-source 134 comprises a container of a cryogenic fluid, for example liquid nitrogen, liquid carbon dioxide, or any other cryogenic compound that can be stored as liquid or gas in high or low pressure in room temperature. Alternatively, the cryo-source 134 comprises a closed-loop internal reservoir, optionally including at least one thermoelectric cooler (TEC).

According to some exemplary embodiments, at least one of the wash source 108, optics 136, cryo source 134 and/or evacuation pump 107 is part of the cryotherapy system 100. Alternatively, at least one of, the wash source 108, optics 136, cryo source 134 and/or evacuation pump 107, are external elements, connectable to the system 100.

### Exemplary Cryotherapy

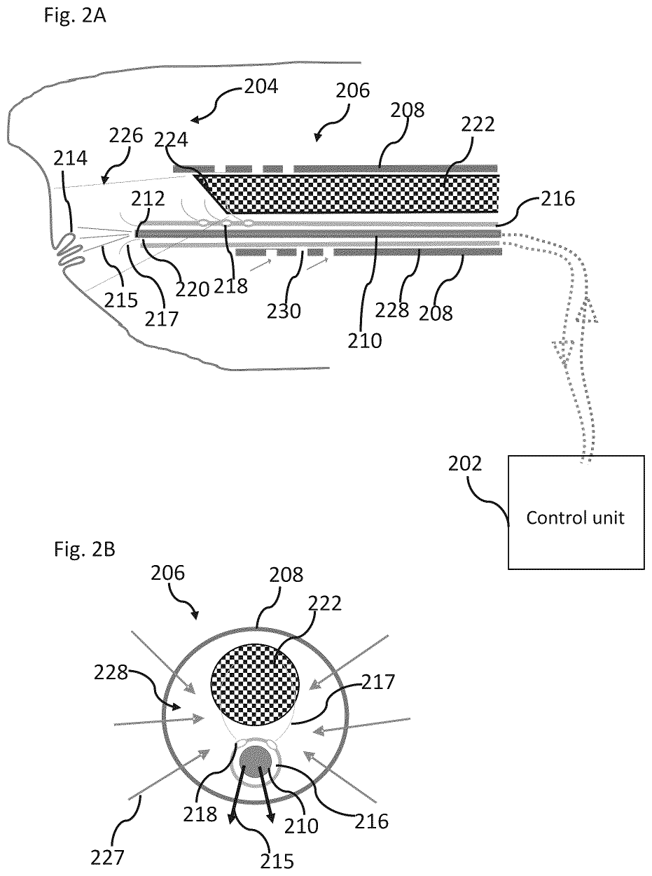

Reference is now made to FIGS. 2A and 2B depicting a cryotherapy device inside a body lumen, according to some exemplary embodiments of the invention.

According to some exemplary embodiments, a cryotherapy device coupled to a control unit 202, for example a control console, is introduced through an opening in the body, for example a surgical opening or an anatomical opening, while the control unit 202 remains outside the body of a subject. In some embodiments, the cryotherapy device is introduced and navigated into a body lumen 204, for example via an opening of the body lumen, for example an anatomical opening or a surgical opening. In some embodiments, the cryotherapy device is introduced and navigated into a body lumen 204 within a working channel of an endoscope or within a rigid or a flexible structure, for example a sleeve 208.

According to some exemplary embodiments, a distal end 206 of the cryotherapy device is introduced into the body lumen. In some embodiments, the distal end 206 comprises at least one cryo fluid outflow channel 210 within an inner lumen of the cryotherapy device, configured to deliver cryo fluid from a cryo fluid source coupled to the control unit 202, to a cryo fluid opening 212. In some embodiments, the cryo fluid opening 212 is configured to release cryo fluid, for example to spray cryo fluid into the body lumen 204. In some embodiments, the cryo fluid is optionally sprayed towards a tissue 214 in a treatment region.

According to some exemplary embodiments, the distal end 206 comprises at least one washing fluid outflow channel 216 within an inner lumen of the cryotherapy device, configured to deliver washing fluid 217 from a washing fluid source coupled to the control unit 202 to a plurality of washing fluid openings, for example openings 218 and 220 at the distal end 206 of the cryotherapy device. In some embodiments, the washing fluid openings are located at different locations along the distal end 206. In some embodiments, at least some of the washing fluid openings are sideways facing openings. Alternatively or additionally at least some of the washing fluid openings are forward facing openings, for example openings facing the treatment region.

According to some exemplary embodiments, the distal end 206 comprises at least one visualization assembly, for example visualization assembly 222. In some embodiments, a distal end 224 of the visualization assembly 222 is located in the body lumen. In some embodiments, the distal end 224 comprises at least one of an aperture, illumination, a windows and a lens, for example a forward facing aperture, a forward facing window, a forward facing lens, configured to allow visualization of a treatment space between the distal end 206 and the target region and/or the tissue 214 in the target region. In some embodiments, at least one of the treatment space, the target region and the target tissue 214 is within a field of view (FOV) 226 of the visualization assembly 222, for example a FOV of the visualization assembly distal end 224. In some embodiments, the distal end 224, for example an aperture at the distal end 224, optionally comprises a lens and/or a transparent window. In some embodiments, the visualization assembly 222 comprises a visualization channel within said cryotherapy device that ends with the aperture 224 at the distal end 206 of the cryotherapy device. In some embodiments, at least some of the washing fluid openings, for example openings 218 are facing and/or directed to the visualization assembly distal end 224, for example to release washing fluid towards the distal end 224, for example to clean at least part of the visualization assembly, for example a lens, illumination end unit, a window or an aperture located at the visualization assembly distal end.

According to some exemplary embodiments, the cryotherapy device comprises at least one evacuation flow path, for example at least one evacuation channel 228, configured to evacuate fluids from the body lumen, for example to a reservoir optionally located outside the body, for example cryo fluid and/or washing fluid. Alternatively or additionally, the evacuation channel 228 evacuates body fluids from the body lumen to the reservoir, or release the evacuated body fluids to room environment outside the body. In some embodiments, the at least one evacuation channel 228 comprises at least one opening at the distal end 206 of the cryotherapy device. In some embodiments, the at least one evacuation opening, for example evacuation openings 230 are openings in the cryotherapy device outer surface configured to evacuate fluid from the body lumen. In some embodiments, the at least one evacuation opening is a forward facing opening, a side opening, or an opening that face at least one of, the tissue 214, a target region, a treatment space, or a side-treatment volume, where unneeded fluids and particles accumulate.

According to some exemplary embodiments, the control unit 202 coupled to the cryotherapy device optionally controls flow through at least one of, the cryo fluid outflow channel 210, the at least one evacuation channel 228, and the at least one washing fluid outflow channel 216, for example as described in international patent application WO2018142411A1. Optionally, the control unit controls flow through at least one of, the cryo fluid outflow channel 210, the at least one evacuation channel 228, and the least one washing fluid outflow channel 216, based on signals received from at least one sensor indicating at least one of, temperature, changes in temperature, pressure and/or changes in pressure, humidity and/or changes in humidity.

According to some exemplary embodiments, for example as shown in FIG. 2B, cryo fluid 215 is released from at least one opening of a cryo fluid outflow channel 210, and washing fluid is released from openings 218 towards the visualization assembly 222, for example towards the visualization assembly distal end 224. In some embodiments, fluids 227 are evacuated from the body lumen through the at least one evacuation channel 228.

### Exemplary Formation of Liquid Drops and/or Frost

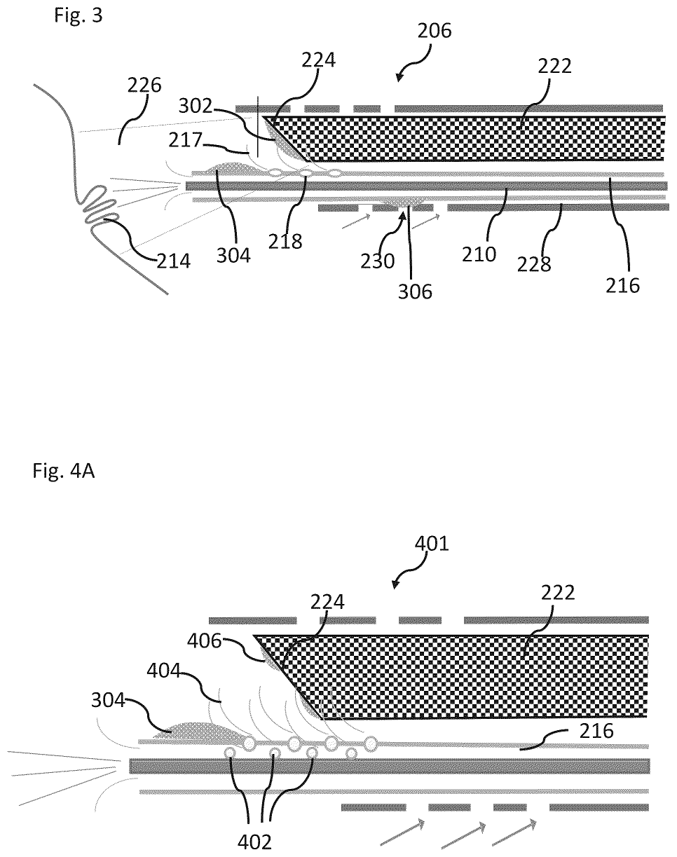

According to some exemplary embodiments, the release of cryo fluid within the body lumen, for example a body lumen that optionally contains body fluids, may cause condensation and the formation of condensed particles, for example the formation of liquid drops. Alternatively or additionally, the release of cryo fluid may freeze body fluid. In some embodiments, formation of liquid drops and/or frost on one or more surfaces of the cryotherapy device, may interfere with evacuation of fluids from the body lumen, with visualization of the treatment space and/or target region and/or may block flow through one or more channels of the cryotherapy device. Reference is now made to FIG. 3 depicting formation of liquid drops and/or frost at one or more locations of a cryotherapy device, according to some exemplary embodiments of the invention.

According to some exemplary embodiments, at least one liquid drop, for example liquid drop 302 is formed on a surface and/or are attached to the surface of the visualization assembly 222, for example to a distal end 224 of the visualization. In some embodiments, the liquid drop 302 is formed and/or attached to an aperture, illumination end unit, a lens and/or a window located at the distal end 224. In some embodiments, formation and/or attachment of the at least one liquid drop 302 to the distal end 224 interfere with the visualization of the target tissue 214 and/or narrows the FOV 226.

According to some exemplary embodiments, at least one liquid drop, for example liquid drop 304 is formed on and/or attached to a surface of the cryotherapy device, for example a surface at the distal end 206 of the cryotherapy device. In some embodiments, the surface is optionally an internal surface of the cryotherapy device, for example a surface of one or more channels and/or lumen of the cryotherapy device. In some embodiments, for formation and/or attachment of the at least one liquid drop 304 optionally interferes with visualization of the target tissue 214, for example if the at least one liquid drop 304 is located within the FOV 226. Alternatively or additionally, formation and/or attachment of the at least one liquid drop 304 to a surface of the cryotherapy device optionally interferes with movement of the distal end 206 within the body lumen and/or with flow in one or more lumen of the cryotherapy device. For example, formation or attachment of the at least one liquid drop 304 on a surface optionally blocks one or more openings of at least one of, at least one cryo fluid outflow channel 210, the at least one washing fluid outflow channel 216, and the at least one evacuation channel 228.

According to some exemplary embodiments, at least one liquid drop, for example liquid drop 306 is formed within an inner lumen of the cryotherapy device, for example an inner lumen of at least one channel of the cryotherapy device located at the distal end 206. In some embodiments, the at least one liquid drop 306 is attached to an inner surface of an inner lumen of the cryotherapy device, for example to an inner surface of at least one channel of the cryotherapy device located at the distal end 206. In some embodiments, the at least one liquid drop 306 is attached to an inner surface of an inner lumen of the at least one of, the evacuation channel 228, the at least one cryo fluid outflow channel 210 and the at least one washing fluid outflow channel 216. In some embodiments, attachment of the at least one liquid drop 306 to an inner surface of a channel optionally blocks flow through the channel, for example optionally blocks evacuation of fluids through the at least one evacuation channel 228.

According to some exemplary embodiments, the at least one liquid drop is in a liquid state or is frozen.

According to some exemplary embodiments, washing fluid is released through one or more openings, for example openings 218 to prevent the formation of the at least one liquid drop and/or the attachment of the at least one liquid drop to at least one surface of the cryotherapy device. Alternatively or additionally, the washing fluid is released through one or more openings, for example openings 218 to break or dry at least one liquid drop, for example a liquid drop attached to at least one surface of the cryotherapy device. In some embodiments, a control unit coupled to the cryotherapy device, for example control unit 202 shown in FIG. 2A or control unit 104 shown in FIG. 1B, signals at least one valve located on the washing fluid flow path to open. Alternatively or additionally, the control unit activates at least one pump coupled to the washing fluid flow path, to deliver washing fluid towards the distal end 206. In some embodiments, the control unit signals the at least one valve and/or activates the at least one pump in response to signals indicating the formation or of at the least one liquid drop and/or frost. Alternatively or additionally, the control unit signals the at least one valve and/or activates the at least one pump in response to signals indicating the attachment and/or accumulation of the at least one liquid drop and/or frost on at least one surface of the cryotherapy device. Alternatively, the control unit signals the at least one valve and/or activates the at least one pump at predetermined time periods or in response to an input signal from a user.

### Exemplary Liquid Droplets Formation Prevention and/or Removal of Liquid Drops

According to some exemplary embodiments, washing fluid is released in order to prevent the formation of the liquid drops and/or frost within the body lumen. In some embodiments, the liquid drops are formed and/or accumulates at regions that are less accessible to the released washing fluid. Additionally, the amount of washing fluid that can be released in the body lumen is limited due to pressure increase and temperature. In some embodiments, flow and pressure limitations result with inability to use unlimited amount of washing fluid. Optionally, potential harmful liquid drops may be built and/or remain in locations where there is no washing fluid flow or where washing fluid flow is not strong enough to dry or break the drops.

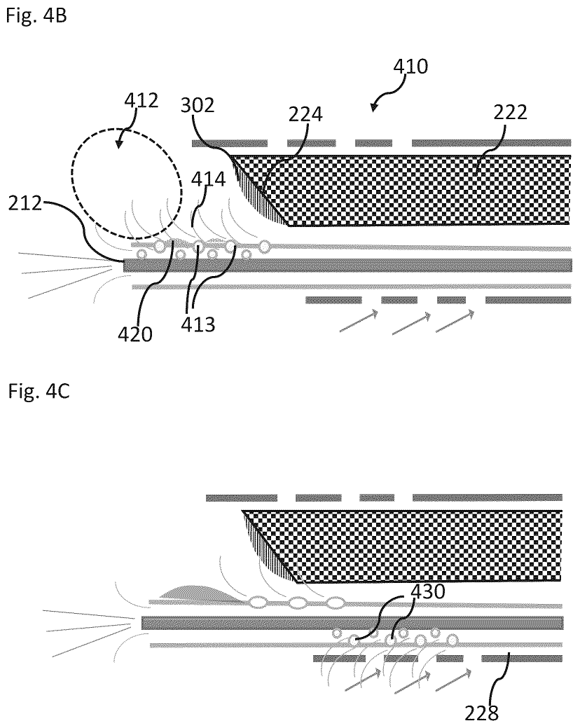

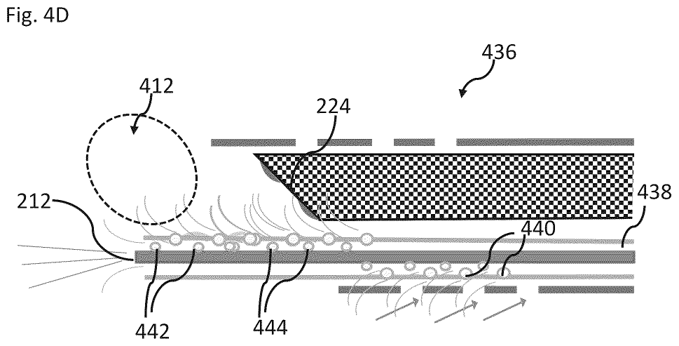

Reference is now made to FIGS. 4A and 4B, depicting a cryotherapy device having a washing fluid outflow channel with a plurality of small openings, according to some exemplary embodiments of the invention.

According to some exemplary embodiments, a cryotherapy device comprises at least one washing fluid outflow channel 216 at a distal end 401 of the cryotherapy device with a plurality of openings 402 configured to release washing fluid 404 inside the body lumen. In some embodiments, the plurality of openings 402 are shaped and sized to direct washing fluid towards a distal end 224 of the visualization assembly 222, for example towards a lens, an aperture, an illumination end unit and/or a window located at the distal end 224.

According to some exemplary embodiments, the plurality of openings 402 have a diameter or width in a range of 0.01 mm-2 mm, for example 0.05 mm-0.5 mm, 0.07 mm-1 mm, 0.8 mm-2 mm or any intermediate, smaller or larger range of values. In some embodiments, the number of plurality of openings 402 is in a range of 3-1000, for example 3-50, 20-100, 50-500, 300-1000 or any intermediate, smaller or larger number of openings. Optionally, the plurality of openings 402 are arranged in an array where a minimal distance between two adjacent openings is in a range of 0.2-5 mm, for example 0.2-2 mm, 1-3 mm, 2-5 mm or any intermediate, smaller or larger range of values.

According to some exemplary embodiments, for example as shown in FIG. 4B, at least some of the plurality of washing fluid openings 413 are positioned distally to the distal end 224 of the visualization assembly. In some embodiments, at least some of the washing fluid openings 413 are optionally positioned proximal to a cryo fluid opening 212 and distal to the distal end 224, for example to generate a flow protecting space 412 between the cryo fluid outflow opening 212 and the distal end 224 of the visualization assembly. In some embodiments, the flow protecting space is created by washing fluid exiting through the plurality of openings 413 located distally to the visualization assembly distal end 224. Optionally, the flow protecting space 412 is created around the opening 212, for example using washing fluid exiting through additional washing fluid openings located opposite to openings 413. In some embodiments, the created flow protecting space 412 prevents or reduces cryo fluid flow, for example cryo fluid mist from flowing towards the visualization assembly, for example from flowing towards a lens or a window located at the distal end 224.

Alternatively or additionally, washing fluid 414 released through the plurality of openings 413 disrupts, clears and/or dries existing liquid drops, for example drop 420, that is attached to at least one surface of the cryotherapy device, at the distal end 410 of the cryotherapy device. Alternatively or additionally, washing fluid 414 released through the plurality of openings 413 disrupts, clears and/or dries existing liquid drops, for example drop 302, that is attached to a lens or a window at the distal end 224 of the visualization assembly. Alternatively or additionally, washing fluid 414 released through the plurality of openings 413 dries an area, a space and/or a volume surrounding at least partly channels of the cryotherapy device, for example at least one cryo fluid outflow channel, at least one washing fluid channel and/or at least one evacuation channel, or at least one opening thereof.

According to some exemplary embodiments, for example as shown in FIG. 4C, the at least one washing fluid channel comprises a plurality of openings, for example openings 430, facing and/or directed to the at least one evacuation channel 228. In some embodiments, washing fluid flow through the openings 430, disrupts and/or dries one or more liquid drops in the at least one evacuation channel 228, for example to prevent blockage of the at least one evacuation channel 228 optionally by frost.

According to some exemplary embodiments, for example as shown in FIG. 4D, at least one washing channel 438 of a cryotherapy device comprises a plurality of washing fluid openings, for example openings 440, 442 and 444 located at different axial positions and/or radial positions along the distal end 436 of the cryotherapy device. In some embodiments, at least some of washing fluid openings, for example openings 442, are located at an axial and/or radial location between a visualization assembly distal end 224, and a cryo fluid opening 212, for example to release and/or direct washing fluid to form the flow protecting space 412. Alternatively or additionally, at least some of the washing fluid openings, for example openings 442 are axially and/or radially positioned along the cryotherapy distal end 436, for example to direct washing fluid flow towards and/or along surfaces of the cryotherapy device distal end.

Alternatively or additionally, at least some of washing fluid openings, for example openings 444 are located at an axial location and/or a radial position that faces at least partly the visualization assembly distal end 224, for example to release and/or direct washing fluid towards a lens or a window located at the distal end 224.

Alternatively or additionally, at least some of the washing fluid openings, for example openings 440 are located at an axial and/or radial position along the cryotherapy distal end 436 facing at least one evacuation channel.

According to some exemplary embodiments, the radial and/or axial position of the washing fluid openings, the size and/or shape of the openings, the number of the openings in an array of openings, and/or a distance between adjacent openings in an array of openings is selected to allow, for example optimization of washing fluid flow, for example amount and/or pressure of washing fluid released into the body lumen. Alternatively or additionally, the radial and/or axial position of the washing fluid openings, the size and/or shape of the openings, the number of the openings in an array of openings, and/or a distance between adjacent openings in an array of openings is selected to prevent formation of liquid drops or frost and/or to disrupt and dry already formed liquid drops.

According to some exemplary embodiments, washing fluid openings, for example openings 442, configured to release and/or direct washing fluid to generate the flow protecting space 412, have a width or diameter in a range of 0.01 mm-2 mm, for example 0.05 mm-0.5 mm, 0.07 mm-1 mm, 0.8 mm-2 mm or any intermediate, smaller or larger range of values.

In some embodiments, the openings 442 are arranged in an array having 3-500 openings, for example 3-50 openings, 30-200 openings, 100-250 openings, 250-500 openings, or any intermediate, smaller or larger range of openings. In some embodiments, the plurality of openings is arranged radially or aligned. In some embodiments, a distance between adjacent openings in the array is fixed. Alternatively, the distance between adjacent openings in the array varies.

According to some exemplary embodiments, washing fluid openings, for example openings 440, configured to release and/or direct washing fluid towards the at least one evacuation channel, have a width or diameter in a range of 0.01 mm-2 mm, for example 0.05 mm-0.5 mm, 0.07 mm-1 mm, 0.8 mm-2 mm or any intermediate, smaller or larger range of values. In some embodiments, the openings 442 are arranged in an array having 5-500 openings, for example 5-50 openings, 30-200 openings, 100-250 openings, 250-500 openings, or any intermediate, smaller or larger range of openings. In some embodiments, the plurality of openings is arranged radially or aligned. In some embodiments, a distance between adjacent openings in the array is fixed. Alternatively, the distance between adjacent openings in the array varies.

According to some exemplary embodiments, washing fluid openings, for example openings 444, configured to release and/or direct washing fluid towards a distal end 224 of the visualization assembly, have a width or diameter in a range of in a range of 0.01 mm-2 mm, for example 0.05 mm-0.5 mm, 0.07 mm-1 mm, 0.8 mm-2 mm or any intermediate, smaller or larger range of values. In some embodiments, the openings 444 are arranged in an array having 3-500 openings, for example 3-50 openings, 30-200 openings, 100-250 openings, 250-500 openings, or any intermediate, smaller or larger range of openings. In some embodiments, the plurality of openings is arranged radially or aligned. In some embodiments, a distance between adjacent openings in the array is fixed. Alternatively, the distance between adjacent openings in the array varies.

### Exemplary Washing Fluid Openings

According to some exemplary embodiments, washing fluid openings, for example washing fluid openings at a distal end of a cryotherapy device are shaped and sized, positioned axially along a longitudinal axis of the cryotherapy device or at least one washing fluid channel and/or have a circumferential distribution, to allow disruption and/or clearance of liquid drops from specific regions of the cryotherapy device. In some embodiments, the specific regions comprise at least one of, at least part of the visualization assembly, the at least one evacuation channel and at least one surface of the cryotherapy device.

According to some exemplary embodiments, the washing fluid openings have the same shape and/or size. Alternatively, at least some or all of the washing fluid openings have a different shape and/or size, and/or are arranged in a random arrangement. In some embodiments, the washing fluid openings are distributed evenly along a circumference of the at least one washing fluid channel. Additionally or optionally, the washing fluid openings are distributed along an entire circumference of the at least one washing fluid channel. Alternatively, the washing fluid openings are distributed along a part of the circumference, for example an arc smaller than 360 degrees, for example smaller than 270 degrees, smaller than 180 degrees, smaller than 90 degrees or any intermediate, smaller or larger value, of the circumference.

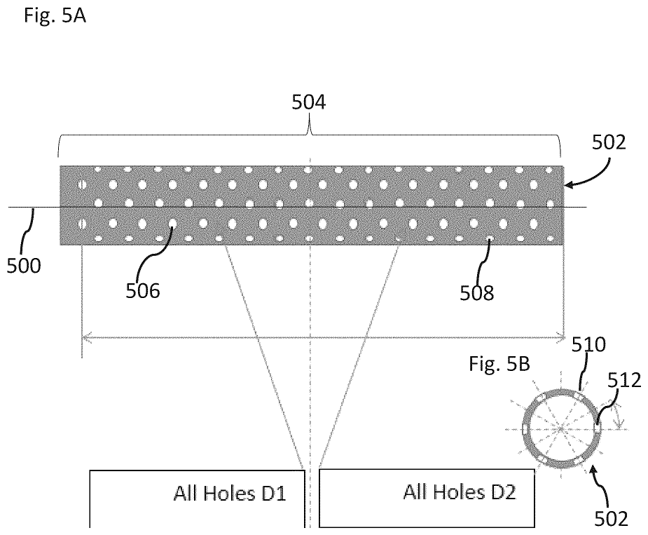

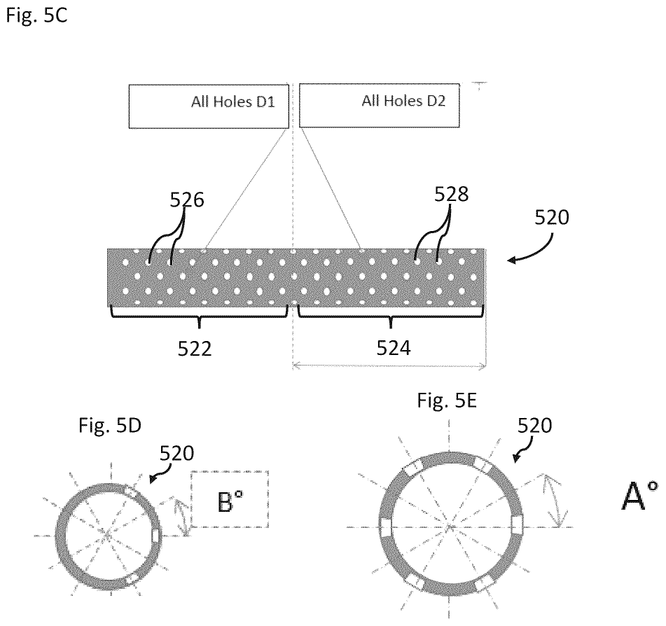

Reference is now made to FIGS. 5A and 5B depicting an arrangement of washing fluid openings distributed evenly on a circumference of at least one washing fluid channel, according to some exemplary embodiments of the invention.

According to some exemplary embodiments, a plurality of openings is distributed axially on a circumference of a washing fluid channel 502, along a longitudinal axis 500 of the washing fluid channel 502. In some embodiments, the plurality of openings are optionally distributed axially along a region having a length 504. Additionally, the plurality of openings are circumferentially distributed along a circumference of the washing fluid channel 502. In some embodiments, for example as shown in FIG. 5B, two adjacent washing fluid openings, for example openings 510 and 512 that have a similar axial location, have an equal circumferential distance. In some embodiments, the washing fluid openings surround entirely the washing fluid channel.

According to some exemplary embodiments, all of the plurality of openings have the same shape and size, for example width or diameter. Alternatively, for example as shown in FIG. 5A, the plurality of openings comprise 2 or more groups of openings with different shape and/or size. For example, a first group of openings 506 have a larger size compared to a second group of openings 508. In some embodiments, for example as shown in FIGS. 5A and 5B, the plurality of openings, for example openings 506 and 508 are distributed evenly, for example have a similar distance between two adjacent openings, on the circumference of the washing fluid channel 502. Alternatively, the shape, size, number, location and/or arrangement of the openings is random.

Reference is now made to FIGS. 5C-5E depicting washing fluid openings with different sized and circumferential distribution, according to some exemplary embodiments of the invention.

According to some exemplary embodiments, washing fluid openings are axially distributed along a circumference of a washing fluid channel 520. In some embodiments, at least some of washing fluid openings, for example openings 522 located axially within region 522 are optionally circumferential distributed only on a portion of the circumference of the washing channel 520, for example as shown in FIG. 5D. In some embodiments, a different group of the washing fluid openings, for example openings 528 located at region 524, is axially spaced apart from the openings 526 of region 522. In some embodiments, openings 528, for example as shown in FIG. 5E are circumferentially distributed along the entire circumference, and optionally surround the washing channel 520.

According to some exemplary embodiments, a washing channel comprises 2 or more types of washing fluid openings, which optionally differ from each other in at least one of, size, shape, width diameter, axial distribution, and circumferential distribution. Additionally or optionally, when the two or more types of washing fluid openings are arranged in separate arrays, or in random order, for example in a single array, they optionally differ in the density of the openings per array, for example the number of openings per area size. In some embodiments, each array optionally has a different axial length along the circumference of the washing fluid.

### Exemplary Impingement

According to some exemplary embodiments, the cryotherapy device comprises a plurality of openings at a distal end of the cryotherapy device that at least partly face a distal end of the visualization assembly. In some embodiments, the plurality of openings face at least partly a lens or a window located at the visualization assembly distal end. In some embodiments, the plurality of openings are located on a circumference of at least one washing fluid channel. Alternatively or additionally, the plurality of openings are located on a circumference of a cryo fluid outflow path. In some embodiments, the plurality of openings are configured for at least one gas jet impingement, flashing, and/or cleaning the visualization assembly from liquids and/or other particles.

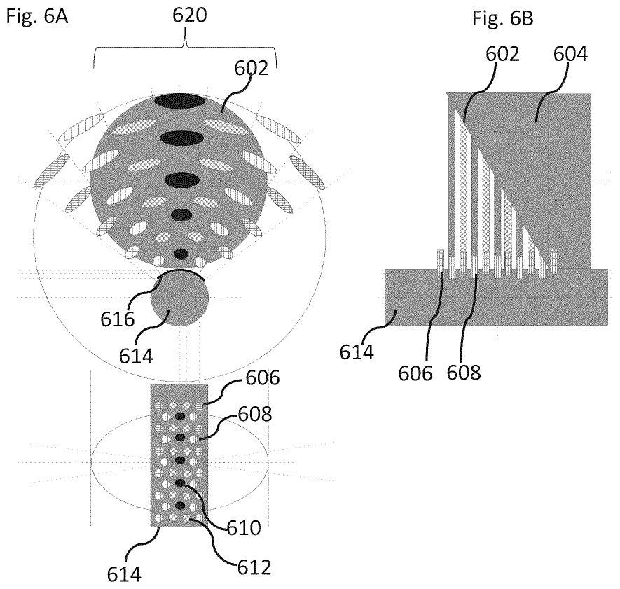

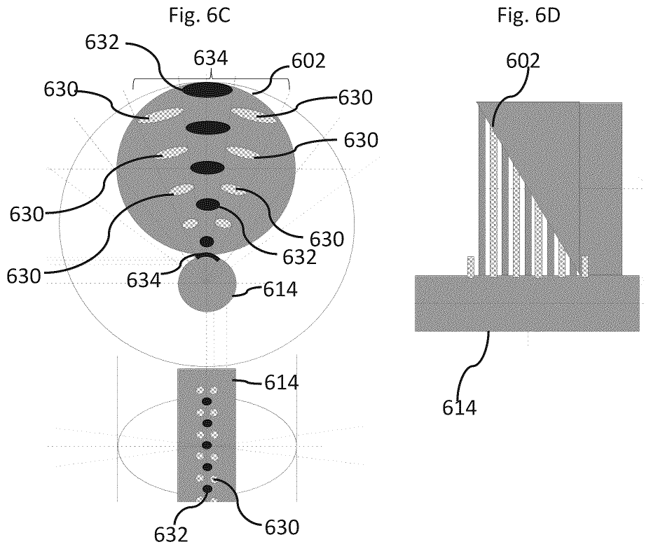

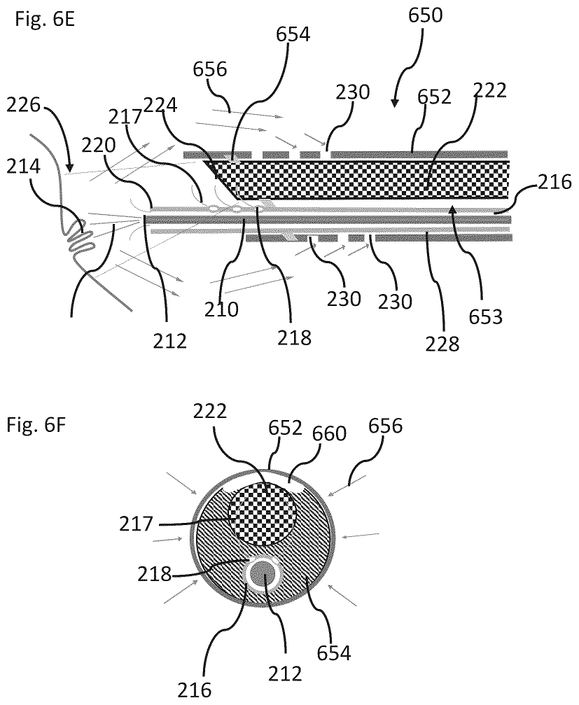

Reference is now made to FIGS. 6A and 6B, depicting openings for gas jet impingement shaped, sized and/or distributed to cover an entire area of a visualization assembly surface, for example a lens, illumination end unit or a window, according to some exemplary embodiments of the invention.

According to some exemplary embodiments, a cryotherapy device comprises a plurality of openings, for example at a distal end of the cryotherapy device, configured to spray jets of fluids, for example gas, onto a surface 602 of a visualization assembly 604. In some embodiments, the surface 602 comprises a lens, illumination end unit or a window. In some embodiments, the plurality of openings, for example openings 606, 608, 610 and 612, are distributed on a circumference of a channel 614. In some embodiments, the channel 614 comprises a washing fluid channel. In some embodiments, the plurality of opening are circumferentially distributed along an arc 616 subtending an angle smaller than 180 degrees, for example an angle smaller than 120 degrees, an angle smaller than 100 degrees, an angle smaller than 90 degrees or any intermediate, smaller or larger value. Alternatively, the openings are circumferentially distributed on a 360 degrees circle surrounding the channel 614. Alternatively, the openings are circumferentially distributed along an arc subtending an angle equal or larger than 180 degrees, for example an angle larger than 200 degrees, an angle larger than 250 degrees, an angle larger than 300 degrees or any intermediate, smaller or larger angle. In some embodiments, a length of the arc is predetermined according to a width or diameter 620 of the visualization assembly surface 602.

According to some exemplary embodiments, at least some of the plurality of openings optionally have a different size and/or shape. Alternatively or additionally, the plurality of openings are distributed on a circumference of the channel 614 as an array. In some embodiments, the density of the openings in the array, for example the number of openings per area, is equal along the entire area of the array. Alternatively, the density of the openings varies, for example between different regions of the array or randomly. In some embodiments, the different size and/or shape of the openings allow, for example different spray cones of liquid and/or gas from each opening type.

According to some exemplary embodiments, the circumferential arc length, the distribution of the openings, the different types of openings and/or the density of the openings is predetermined or selected to cover a selected area of the surface 602, for example to cover an entire area of the surface 602.

Alternatively, for example as shown in FIGS. 6C and 6D, the openings, for example openings 630 and 632, are distributed on the circumference of the channel 614 along an arc which subtends an angle 634 which is smaller than angle 616. In some embodiments, fluid, for example gas released from the openings 630 and 632 cover less than 90%, for example less than 70%, less than 60%, less than 50% or any intermediate, smaller or larger percentage value of the surface 602. In some embodiments, fluid, for example gas released from the openings 630 and 632 towards the surface 602 forms a clearing zone 634 having an area which is smaller than an area of the surface 602. In some embodiments, this allows to optimize jet strength and/or gas amount while clearing a major portion, for example up to 80%, up to 70%, up 70 50% of a FOV between the surface 602 and a cryo fluid treated tissue.

### Exemplary Seal

According to some exemplary embodiments, evacuation fluid flow is directed using one or more seals, shaped, sized and/or positioned in the cryotherapy device distal end to direct fluid to openings of the at least one evacuation channel. Reference is now made to FIGS. 6E and 6F, depicting a cryotherapy device having a distal end with at least one seal, for example a front seal, according to some exemplary embodiments of the invention.

According to some exemplary embodiments, a distal end 650 of a cryotherapy device comprises an outer sleeve 652 defining an inner channel 653 having a distal end, for example a rigid or flexible distal end introducible into a body lumen, for example a hollow organ. In some embodiments, the cryotherapy device comprises a visualization assembly 222 within the channel 653 having a distal end 224 at a distal end 650 of the cryotherapy device. Additionally, the cryotherapy device comprises at least one cryo fluid outflow channel 210 traveling within said channel 653, and has one or more openings 212 at the cryo fluid distal end 650, optionally facing the target tissue 214. Optionally, the one or more openings 212 are located distally to the visualization distal end 224, for example to allow spraying of cryo fluid towards the target tissue at a distance from the optics that is sufficient to prevent formation of drops on a surface of the visualization at the visualization distal end 224.

According to some exemplary embodiments, the cryotherapy device comprises at least one washing fluid outflow channel 216 within said channel 653. In some embodiments, the washing fluid channel 216 comprises one or more openings, for example opening 218 on a circumference of the channel 216, optionally facing the visualization assembly 222, for example the visualization assembly distal end 224. In some embodiments, the one or more openings 218 are configured to direct washing fluid 217 towards the distal end 224, for example towards a surface of the visualization assembly at the distal end 224. Alternatively or additionally, the one or more openings 218 are configured to direct washing fluid 217 distally to the distal end 224, for example to prevent cryofluid flow towards a surface of the visualization assembly at the distal end 224. Additionally or alternatively, the washing fluid channel 216 comprises one or more forward facing openings, for example opening 220, shaped and sized to direct washing fluid towards the target region 214 and/or the FOV 226. Optionally, opening 220 surrounds openings 212. Optionally, the washing fluid channel 216 and the cryo fluid channel are coaxial. Optionally, the washing fluid channel 216 surrounds the cryo fluid channel 210.

According to some exemplary embodiments, the cryotherapy device comprises at least one evacuation channel, for example evacuation channel 228, configured to evacuate fluid and particles from within the body cavity, for example hollow organ. In some embodiments, a distal end of the evacuation channel 228 is located at the distal end 650 of the cryotherapy device, and a proximal end of the evacuation channel 228 is located outside the body cavity, for example outside the body. In some embodiments, one or more openings within the sleeve 652, for example openings 230, on a circumference of the sleeve 652, are shaped and sized to allow entry of the fluid and particles from within the body lumen into the evacuation channel 228. Additionally, the evacuation channel comprises one or more forward facing openings, optionally facing the target region 214, configured to allow entry and evacuation of fluid and particles into the evacuation channel 228. In some embodiments, at least one wall of the evacuation channel 228 is optionally formed by the sleeve 652.

According to some exemplary embodiments, for example as shown in FIG. 6E, the distal end 650 of the cryotherapy device comprises a seal, for example seal 654, configured to block at least part of a forward evacuation of fluid and particles into the evacuation channel 228. In some embodiments, the seal 654 is located distally to the one or more openings in the outer sleeve 652, for example opening 230. In some embodiments, the seal 654 is positioned proximally to the visualization assembly distal end 224, for example between the visualization assembly distal end 224 and the one or more openings 230.

According to some exemplary embodiments, the seal 654 allows, for example, to direct a flow of fluid 656 and particles to surround the visualization assembly distal end 224 and to penetrate into the evacuation channel through the one or more openings in the circumference of the sleeve 652, proximal to the sleeve 652 and into the evacuation channel 228. A potential advantage of evacuating fluid and particles through one or more openings proximal to the visualization assembly distal end may be to keep away mist and cold from the visualization assembly, for example from the optics. In some embodiments, the seal 654 comprises at least one opening, for example opening 660, configured to allow passage and evacuation through the seal 654 of at least 5%, for example at least 10%, at least 30%, at least 50%, at least 70% or any intermediate, smaller or larger percentage of fluid and particles, into the evacuation channel 228, relative to an unblocked, open evacuation channel. In some embodiments, the seal 654 blocks passage of at least 30%, for example at least 40%, at least 50%, at least 60%, at least 70%, at least 80%, at least 95% or any intermediate, smaller or larger percentage of fluid and particles into the evacuation channel 228.

According to some exemplary embodiments, the at least one opening 660 in the seal 654 is located between a surface of the visualization assembly 222 and an inner surface of the sleeve 652. A potential advantage of having an opening in the seal may be to allow evacuation from the body lumen in case openings in the circumference of the sleeve, for example evacuation openings into the evacuation channel are clogged or closed.

### Exemplary Internal Reducer

According to some exemplary embodiments, the cryotherapy device comprises at least one internal reducer within a washing fluid flow path. In some embodiments, the at least one internal reducer control the amount and/or pressure of washing fluid directed towards the plurality of washing fluid openings. In some embodiments, controlling the amount and/or pressure of washing fluid directed towards the washing openings allows, for example, to control a shape and/or size of a spray cone or a clearing zone formed by washing fluid released from the washing openings.

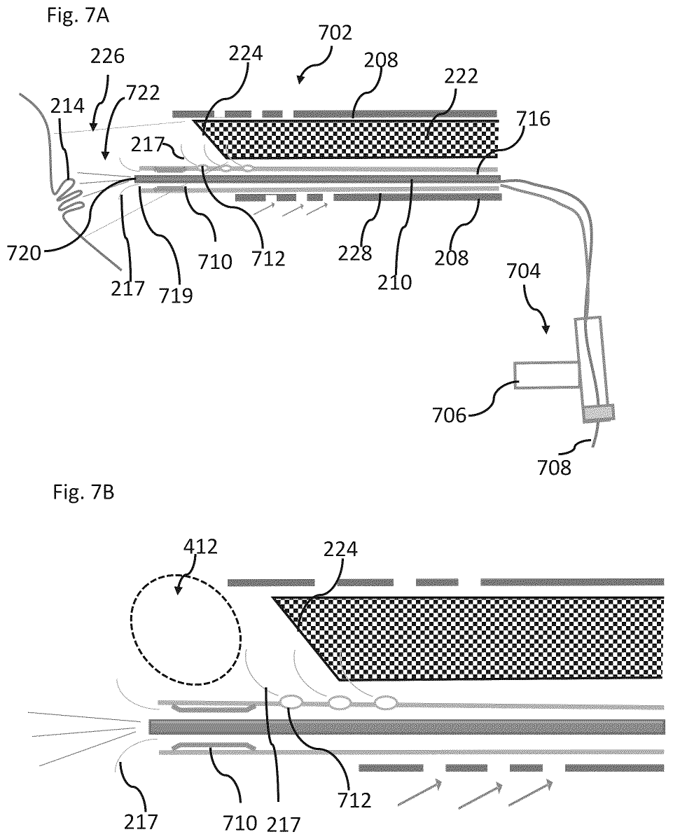

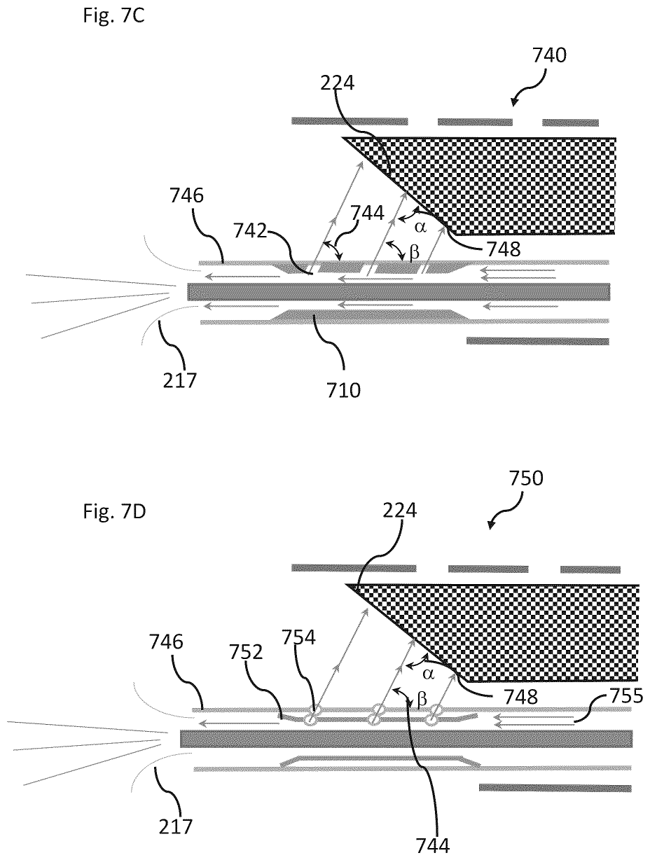

Reference is now made to FIG. 7A, depicting a distal end of a cryotherapy device, for example distal end 702, in which washing fluid directed towards the cryo fluid affected region, for example cryo wash, and a washing fluid directed towards the visualization assembly, are delivered via the same flow path, for example the same channel. In some embodiments, high pressure cryo fluid is introduced into a cryo fluid outflow path, for example a cryo fluid channel, while low pressure washing fluid is introduced into at least one washing fluid channel. In some embodiments, for example as shown in FIG. 7A, the cryotherapy device comprises a connector 704 having a first inlet 706 for low pressure washing fluid, and a second inlet 708 for high pressure cryofluid. In some embodiments, connector 704 allows, for example coaxial flow cryo fluid and washing fluid, for example in at least two coaxial channels. In some embodiments, the washing fluid channel is coaxial and surrounds the cryofluid channel, along at least 50%, for example at least 70%, at least 90% of a length of the cryofluid channel. A potential advantage of having a coaxial arrangement of a washing fluid channel and a cryofluid channel may be to reduce space and/or volume of the device, to increase strength (e.g. in the case of sensitive tubes, like cryo nozzle), to allow keeping the cryo channel in a target temperature by isolating the cryo channel from the body using the washing fluid channel, and to allow radial flow around the cryo nozzle, for example to prevent mist and/or to push mist away from the optics.

According to some exemplary embodiments, for example as shown in FIGS. 7A and 7B, the at least one washing fluid channel 716 comprises at least one internal reducer 710 within the internal lumen of the channel 716. In some embodiments, the internal reducer 210 is configured to divert at least some of washing fluid towards washing fluid openings, for example openings 712 facing at least partly the visualization assembly, for example the optics assembly. In some embodiments, the internal reducer 710 diverts at least some of the washing fluid towards optics wash openings 712, while the rest of the washing fluid flows towards at least one cryo wash opening 719. In some embodiments, washing fluid 217 released from the cryo wash opening 719 is delivered to clear a therapeutic space 722 between a distal end 702 and the treated tissue 214 and/or to clear FOV 722. Additionally, optics washing fluid, for example washing fluid 217 is delivered to at least one of, clear a lens, disrupt liquid particles on the lens and/or to form a flow protecting space 412.

According to some exemplary embodiments, for example as shown in FIG. 7C, a cryotherapy device 740 comprises at least one internal reducer 710 having integrated channels and/or openings, for example channel 742. In some embodiments, the integrated channels and/or openings, for example channel 742 is configured to direct washing fluid towards a selected direction. In some embodiments, for example as shown in FIG. 7C, the channel 742 is oriented at a deflection angle 744 relative to an external surface 746 of the washing fluid channel facing the visualization assembly distal end 224. In some embodiments, a number of channels and/or openings towards the visualization assembly and/or an inner width or diameter of each channel is predetermined, for example to reach a specific impingement angle 748 relative to a surface of a lens or a window at the visualization assembly distal end 224. In some embodiments, the deflection angle of the channel, for example channel 742 is an angle in a range between 1° and 90°, for example between 1° and 40°, between 1° and 20°, between 30° and 60°, between 45° and 90°, or any intermediate, smaller or larger range of values.

According to some exemplary embodiments, for example as shown in FIG. 7D, a cryotherapy device 750 comprises at least one internal reducer 752 within an inner lumen of at least one washing fluid channel 755. In some embodiments, the washing fluid channel 755 comprises a plurality of openings arranged on the circumference of the channel 755, optionally as an array. In some embodiments, the openings, for example openings 754, are shaped and sized and/or are oriented to release washing fluid at a deflection angle 744 relative to an external surface 746 of the washing channel. Alternatively or additionally, the openings, for example openings 754, are shaped and sized and/or are oriented to spray washing fluid at a deflection angle that will generate a specific impingement angle 748.

According to some exemplary embodiments, the openings, for example openings are angled openings, configured to release washing fluid at a specific deflection angle 744, or to reach a specific impingement angle 748. In some embodiments, having an internal reducer 752 and a plurality of openings 754, optionally arranged as an array, allows, for example efficient control on the amount of washing fluid directed towards the visualization assembly, for example efficient control on the optics wash. In some embodiments, for example as shown in FIG. 7D, the internal reducer 752 separating the optics wash from the cryo wash 217 is combined with adjustable directional jets, for example openings 754 which are shaped sized and/or oriented to reach a specific deflection angle 744 and/or a specific impingement angle 748.

According to some exemplary embodiments, for example as shown in FIGS. 7C-7D, positioning a flow reducer in a flow channel, for example a flow reducer with one or more channels directed to washing fluid openings and/or with washing fluid openings, may generate negative pressure or reduced positive pressure within the washing fluid channel, near the one or more channels and/or openings, Optionally leading to a venturi effect. In some embodiments, the negative pressure generates suction through the one or more channels and/or openings, from the outside into the washing fluid channel.

In some embodiments, positioning of the at least one flow reducer within the washing fluid channel, narrows at least a portion of the channel and increases flow speed within the narrowed portion. In some embodiments, the increase in flow speed generates a venturi effect within the narrowed portion, optionally reducing positive pressure or generating negative pressure within the narrowed portion. In some embodiments, the reduction in pressure generates suction force through washing fluid openings in the narrowed region, optionally leading to suction of one or more drops accumulated on surfaces outside the washing fluid channel, for example on at least one surface of the device within the hollow organ, and/or on at least one surface of the visualization assembly.

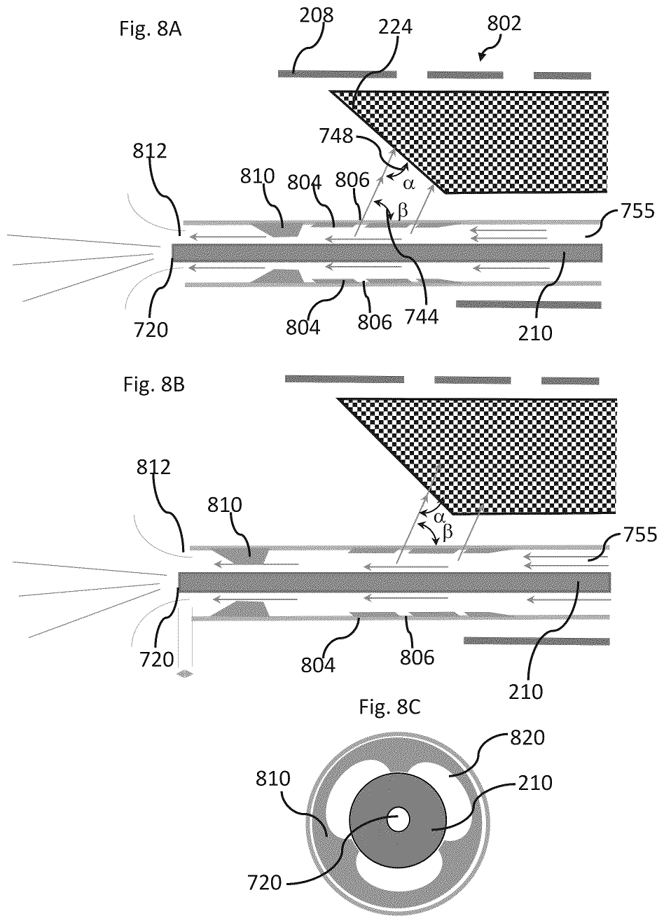

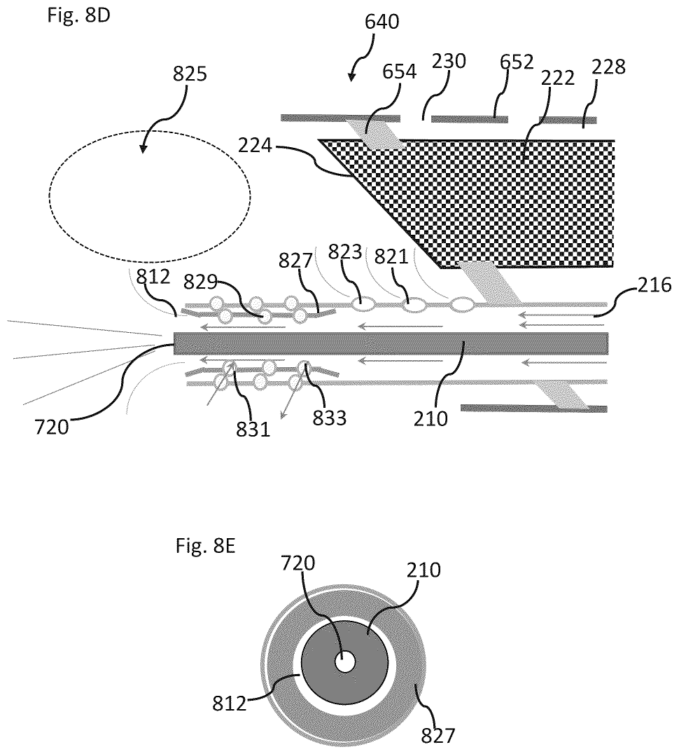

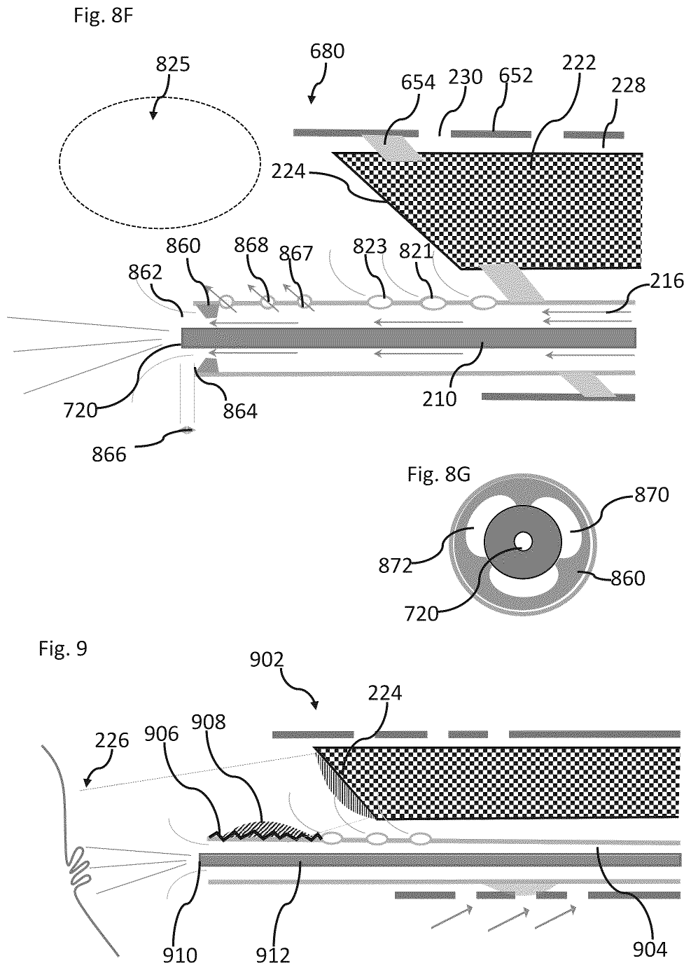

According to some exemplary embodiments, for example as shown in FIG. 8A, a distal end of a cryotherapy device, for example distal end 802, comprises a washing fluid channel 755 and a cryofluid channel 210. Optionally, the washing fluid channel 755 and the cryofluid channel 210 are coaxial, for example the washing fluid channel 755 surrounds, at least partly the cryofluid channel 210. In some embodiments, the washing fluid channel comprises one or more internal flow reducers, for example flow reducer 804 within an inner lumen of the washing fluid channel 755. In some embodiments, the flow reducer 804 is attached to an inner surface washing fluid channel, optionally at least partly surrounding the inner lumen of the washing fluid channel 755. In some embodiments, the flow reducer 804 is shaped as a ring, a tube or an arc smaller than 360 degrees, for example smaller than 270 degrees, smaller than 180 degrees or any intermediate, smaller or larger value, attached to the inner surface of the washing fluid channel 755.

According to some exemplary embodiments, the flow reducer comprises one or more openings, for example openings 806, shaped and sized to deliver washing fluid outside from the washing fluid channel. In some embodiments, the openings 806 are aligned, and/or are fluidically connected, with openings in the wall of the washing fluid channel 755. In some embodiments, the openings 806 are positioned to direct washing fluid towards a surface of the visualization assembly, for example a surface at the distal end 224 of the visualization assembly. Alternatively or additionally, the openings 806 are configured to direct washing fluid towards a space located distally to the distal end 224 and/or to a space between the washing fluid channel outer surface and the visualization assembly distal end 224.

According to some exemplary embodiments, the internal flow reducer, for example flow reducer 810 narrows an inner lumen of the washing fluid channel, optionally leading to an increase in flow speed within the washing flow channel. In some embodiments, the increased flow speed reduces the pressure within the openings 806, optionally leading to suction of fluid and particles into the washing fluid channel 755 through the openings 806.

According to some exemplary embodiments, for example as shown in FIG. 8A, the washing channel comprises at least one additional flow reducer 810, located distally to the flow reducer 804 and/or the openings 806. In some embodiments, the at least one flow reducer 810 is configured to adjust the flow pressure and/or speed of washing fluid exiting through a distal opening 812 of the washing fluid channel 755. Optionally, the distal opening 812 if a forwardly facing opening, for example facing a treatment target. Optionally, the distal opening 812 is adjacent to a distal opening 720 of the cryofluid channel 210. Optionally, the washing fluid channel distal openings 812 at least partly surrounds the cryofluid channel distal opening 720.

According to some exemplary embodiments, position of the at least one flow reducer 810 relative to the washing fluid channel openings 806, controls the washing fluid pressure level near the openings, and/or the pressure level generated within the washing fluid channel 755 near the openings 806. In some embodiments, for example as shown in FIG. 8B, positioning the flow reducer 810 close to the washing fluid openings 806, for example at a distance of up to 5 cm, up to 3 cm, up to 1 cm, or any intermediate, smaller or larger distance from the openings 806 increases the pressure within the washing fluid channel 755 proximal to the flow reducer 810, for example near the openings 806.

According to some exemplary embodiments, for example as shown in FIGS. 8B and 8C, the flow reducer 810 is located near the distal opening 812 of the washing fluid channel, for example at a distance smaller than 5 cm, smaller than 1 cm, smaller than 0.5 cm or any intermediate, smaller or larger distance from the distal opening 812. In some embodiments, the reducer 810 is shaped and sized to control the flow and/or direction of washing fluid exiting from the distal opening 812. Optionally, the reducer 810 controls the flow, for example pressure, and/or direction of the washing fluid exiting from the distal opening 812 relative to the cryofluid sprayed towards a target region from the distal opening 720. In some embodiments, for example as shown in FIG. 8C, the reducer 810 comprises at least one opening 820, for example 2, 3, 4 or larger number of openings, which are shaped and sized to control the pressure and/or direction of washing fluid exiting from the distal opening 812. In some embodiments, the reducer 810 reduces an inner lumen of the washing fluid channel 755 in at least 10%, for example at least 30%, at least 50%, at least 70%, at least 90% or any intermediate, smaller or larger percentage.

### Exemplary Device with an Evacuation Seal and a Flow Reducer

Reference is now made to FIGS. 8D-8G depicting cryotherapy device having at least one washing fluid flow reducer and at least one fluid flow evacuation seal, according to some exemplary embodiments of the invention.

According to some exemplary embodiments, for example as shown in FIGS. 8D and 8E, a cryotherapy device distal end 640 is configured to be introducible into a body lumen, for example a hollow organ. In some embodiments, the cryotherapy device comprises at least one washing fluid channel 216 and at least one cryofluid channel 210. Optionally, the washing fluid channel 216 and the cryofluid channel 210 are coaxial. Optionally or additionally, the washing fluid channel 216 surrounds at least partly the cryofluid channel 210.

According to some exemplary embodiments, the washing fluid channel 216 comprises at least one opening, for example a plurality of openings 821 and 823 through a wall of the washing fluid channel 216. In some embodiments, the openings 821 and 823 are positioned in the washing fluid channel wall at a location facing at least one surface of a visualization assembly 222. In some embodiments, the openings 821 and 823 face at least one surface at a distal end 224 of the visualization assembly. Alternatively or additionally, the openings are located in the washing fluid channel wall to face a space 825 distal to visualization assembly distal end 224. In some embodiments, the openings are configured to direct washing fluid towards the visualization assembly distal end, for example to prevent formation of liquid drops of at least one surface of the visualization assembly and/or to dry and/or to disrupt one or more existing drops.

According to some exemplary embodiments, the washing fluid channel 216 comprises at least one flow reducer, for example flow reducer 827 located at a distal end of the washing fluid channel 216. In some embodiments, the flow reducer 827 comprises at least one opening, for example a plurality of openings 829, and 833, configured to deliver washing fluid through the flow reducer 827 and openings in the wall of the washing fluid channel 216. In some embodiments, for example as described previously in FIGS. 7C and 7D, flow within a narrowed region within the washing fluid channel may generate negative pressure and suction through one or more side openings within the flow reducer, for example opening 831. In some embodiments, an increase flow speed within the narrowed region of the flow reducer lumen generates a venturi effect, which reduces pressure within the flow reducer. Optionally the reduced pressure generates the suction force, for example through the opening 831. In some embodiments, the flow reducer openings are aligned with openings in the washing fluid channel wall, optionally located on the circumference of the washing fluid channel 216. Alternatively, the flow reducer openings comprise openings in the wall of the washing fluid channel 216.

According to some exemplary embodiments, at least some of the flow reducer openings are configured to direct washing fluid to a space located proximally to the cryofluid distal opening 720, and optionally between the distal opening 720 and the visualization assembly distal end 224. Alternatively or additionally, at least some of the flow reducer openings are configured to direct washing fluid to a space surrounding at least partly the washing fluid channel, for example to prevent attachment and/or formation of at least one liquid drop on an external surface of the washing fluid channel. Alternatively or additionally, at least some of the flow reducer openings are configured to apply suction, for example on fluid and/or particles in the body lumen, into the washing fluid channel 216.

According to some exemplary embodiments, the flow reducer 827 is an elongated reducer, having an axial length of at least 0.5 mm, for example at least 1 mm, at least 5 mm or any intermediate, smaller or larger length along a longitudinal axis of the washing fluid channel 216. In some embodiments, the flow reducer 827 shapes the distal opening 812 of the washing fluid channel. In some embodiments, the flow reducer shapes the opening 812 into a ring shape surrounding the cryofluid channel 210, for example as shown in FIG. 8E. In some embodiments, the ring-shaped opening 812 has an equal width. Alternatively, the ring-shaped opening 812 has a variable width.

According to some exemplary embodiments, the cryotherapy device comprises at least one evacuation flow path, for example an evacuation channel 228 configured to remove fluid and/or particles from the body lumen, through at least one evacuation opening, for example evacuation opening 230 in an outer sleeve 652 of the cryotherapy device. In some embodiments, for example as described in FIGS. 6E and 6F, the cryotherapy device comprises at least one evacuation seal 654 at the distal end 650, configured to block in at least 30%, for example at least 50%, at least 70%, at least 90% or any intermediate, smaller or larger percentage value of evacuation fluid flow through a distal, forward opening of the evacuation channel 228. In some embodiments, the evacuation seal 654 is positioned distally to the evacuation opening 230. Optionally, the evacuation seal 654 is positioned between a distal end 224 of the visualization assembly 222, optionally comprising an aperture and/or a lens, and the evacuation opening 230. Optionally, the seal 654 completely blocks evacuation through a distal, forward opening of the evacuation channel 228.

According to some exemplary embodiments, for example as shown in FIGS. 8F and 8G, the washing fluid channel of cryotherapy device distal end 680, comprises at least one flow reducer 860 at a distal end of the washing fluid channel. In some embodiments, the flow reducer 860 is solid. In some embodiments, the flow reducer 860 is short and has an axial length of less than 10 mm, for example less than 5 mm, less than 1 mm or any intermediate, smaller or larger length. In some embodiments, the flow reducer 860 comprises at least one opening, for example openings 870 and 872, through which washing fluid forwardly exits from the washing fluid channel. In some embodiments, the openings 870 and 872 are spaced apart. Alternatively, the openings 870- and 872 are interconnected. In some embodiments, the openings 870 surround at least one distal opening 720 of the cryofluid channel 210, for example as shown in FIG. 8G.

According to some exemplary embodiments, the flow reducer 860 is configured to control and/or direct washing fluid exit from the distal opening 862 of the washing fluid channel. In some embodiments, the distal opening 862 is located at a distance 866 proximal to the cryofluid distal opening 720.

According to some exemplary embodiments, the flow reducer 860 is configured to separate, and keep a fixed distance between the at least one cryo fluid outflow channel 210, and walls of the at least one washing fluid outflow channel 216. In some embodiments, maintaining the fixed distance allows, for example, to heat transfer between the cryo fluid outflow channel 210 and the washing fluid outflow channel 216. Additionally or alternatively, the flow reducer 860 is configured to fix the opening 862 of the washing outflow channel 216 and the cryo fluid opening 720 in a predetermined longitudinal distance 866. In some embodiments, the longitudinal separation distance 866 is in a range of 0-2 mm, for example 0-0.2 mm, 0.1-0.5 mm, 0.4-1 mm, 0.5-2 mm or any intermediate, smaller or larger value.

According to some exemplary embodiments, the washing fluid channel 216 comprises at openings in the channel wall facing the visualization assembly distal end 224, for example openings 821 and 823. In addition, the washing fluid channel comprises one or more additional openings in the channel wall, for example opening 868, configured to direct washing fluid to a space between the cryofluid channel distal opening 720 and the visualization assembly distal end 224. In some embodiments, washing openings 821 are shaped and sized and/or positioned to direct washing fluid towards a surface of the visualization assembly, for example illumination and/or a lens optionally located at distal end 224. In some embodiments, the washing fluid directed via openings 821 and 823 pushes particles and/or drops away from the visualization assembly surface. In some embodiments, openings 867 and 868 are shaped and sized and/or positioned to direct washing fluid towards space 725 where mist and/or gasses accumulate.

### Exemplary Pre-Treated Surface

According to some exemplary embodiments, in order to prevent adhesion and/or formation of at least one liquid drop to at least one surface of the cryotherapy device, the surface is pretreated, for example coated to acquire a rough texture. Alternatively or additionally, the surface is pretreated, for example to reduce size and/or shape of drops, optionally by directing liquids to form shallow layers instead of drops. Reference is now made to FIG. 9, depicting a distal end of a cryotherapy device having at least one channel with a rough exposed external surface, according to some exemplary embodiments of the invention.

According to some exemplary embodiments, a cryotherapy device distal end 802 comprises at least one surface which is exposed to an environment of a treated body lumen, for example surface 906. In some embodiments, the exposed surface 906 is an external surface of at least one washing fluid channel 904. In some embodiments, at least a region of the exposed surface 906 is pretreated, for example precoated, or is manufactured to acquire a rough surface 906. In some embodiments, the exposed rough surface is optionally located between a visualization assembly distal end 224, and an opening 910 of a cryo fluid outflow channel 912. Additionally or alternatively, the exposed rough surface is located within the FOV 226. In some embodiments, the generated rough surface 906 optionally reduces reflection of light within the FOV 226 compared to a smooth surface.

According to some exemplary embodiments, in order to generate the rough surface 906. the surface is coated with hydrophilic or hydrophobic coatings, sandblasting, electro etching or other surface thermal treatments. In some embodiments, the surface is coated with chemical, electro-chemical and/or mechanical treatments.

### Exemplary Visualization Assembly with an Angled Distal End

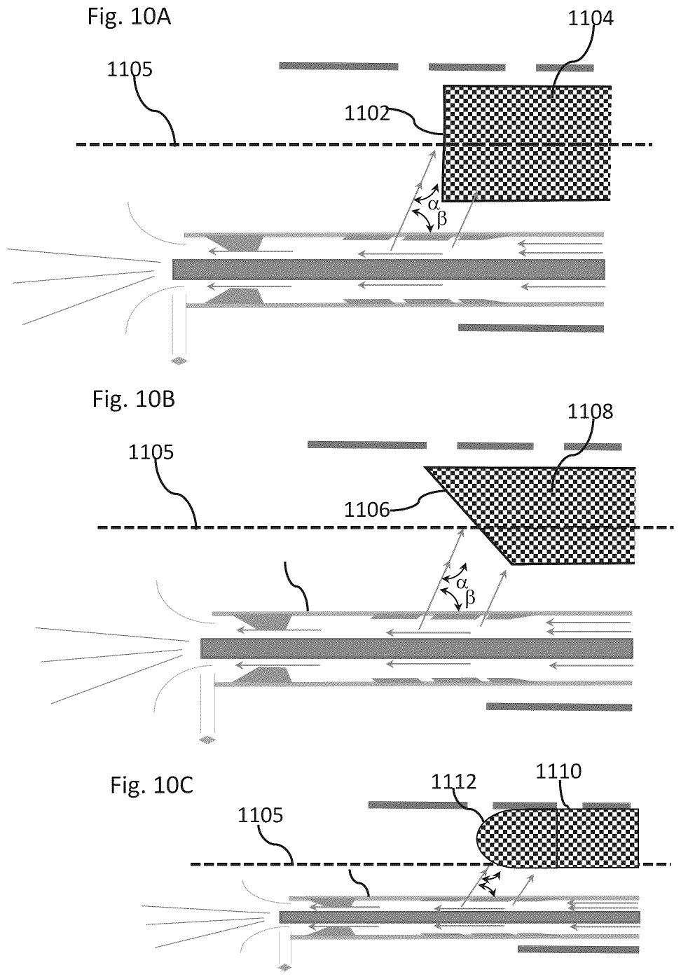

Reference is now made to FIGS. 10A-10C, depicting a visualization assembly with a planar, an angled, or a curved surface at the distal end, according to some exemplary embodiments of the invention.

According to some exemplary embodiments, a visualization assembly comprises a surface at a distal end of the visualization assembly that faces a body cavity, for example a target region at the body cavity. In some embodiments, for example as shown in FIG. 10A, a surface 1102 at a distal end of the visualization assembly 1104 has an angle of about 90 degrees relative to a longitudinal axis 1105, for example a long axis, of the cryotherapy device. Alternatively, for example as shown in FIG. 10B, an angled surface 1106 at a distal end of the visualization assembly 1108, has an angle smaller than 90 degrees, for example smaller than 50 degrees, smaller than 45 degrees, smaller than 20 degrees, in a range between 90 and 5 degrees, in a range between 50 and 30 degrees, relative to the axis 1105. In some embodiments, a slope of the angle surface is a forward, for example an inwardly facing, slope. Alternatively, a surface at the distal end of the visualization assembly has reverse, for example an outwardly facing, slope.

According to some exemplary embodiments, for example as shown in FIG. 10C, a surface 1112 at a distal end of the visualization assembly 1110, is curved.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A cryotherapy system, comprising,
an elongated cryotherapy device having an elongated body, a proximal end and a distal end, wherein said device is shaped and sized to be positioned inside a hollow organ, comprising:
a cryo inflow path inside said elongated body configured to allow cryogenic flow from a cryogenic fluid source into said hollow organ;
a washing inflow path inside said elongated body comprising at least one sideways washing opening in a wall of said washing inflow path, wherein said washing inflow path fluidically connects a washing fluid source with said at least one sideways washing opening and configured to release washing fluid within said hollow organ;
at least one flow reducer positioned within said washing inflow path distally to said at least one sideways washing opening and blocks enough lumen to increase washing fluid flow through said at least one sideways opening.

2. A system according to claim 1, wherein said at least one flow reducer blocks at least 10% of an inner lumen of said washing inflow path located distally to said at least one sideways washing opening.

3-6. (canceled)

7. A system according to claim 1, wherein said washing inflow path comprises at least one distal washing opening at a distal end of said washing inflow path configured to release washing fluid into a treatment space within said hollow organ located distally to said elongated cryotherapy device.

8. A system according to claim 7, wherein said at least one flow reducer is positioned within said washing inflow path proximally to said at least one distal washing opening.

9. A system according to claim 7, wherein said at least one distal washing opening is an opening in said at least one flow reducer.

10. A system according to claim 9, wherein said cryo inflow path comprises at least one distal opening configured to release cryofluid into said treatment space and/or towards a treatment target within the hollow organ located distally to said elongated cryotherapy device, and wherein said at least one distal washing opening surrounds at least partly said cryo inflow path distal opening.

11. A system according to claim 1, wherein said at least one flow reducer contacts an inner surface of said washing inflow path and/or an outer surface of said cryo inflow path.

12. A system according to claim 11, wherein said at least one flow reducer is shaped as a ring, an arc contacting the inner surface of said washing inflow path and/or an outer surface of said cryo inflow path.

13. (canceled)

14. A system according to claim 57, wherein said at least one sideways washing opening comprises a plurality of openings axially and/or angularly distributed in a portion of a wall of said washing inflow path facing said optics assembly and/or said FOV.

15. A system according to claim 1, wherein said at least one sideways washing opening comprises a plurality of openings axially and/or angularly distributed in a portion of a wall of said washing inflow path at least partly surrounding said washing inflow path inner lumen.

16. A system according to claim 15, wherein said plurality of openings have different shapes and/or sizes.

17. A system according to claim 15, wherein said plurality of openings are evenly distributed in said wall portion.

18. A system according to claim 15, wherein said plurality of openings are distributed in varying densities of openings per area in said wall portion.

19-20. (canceled)

21. A system according to claim 1, wherein said cryotherapy device comprises at least one fluid evacuation flow path inside said elongated body having at least one first evacuation opening at a distal end of said at least one fluid evacuation flow path, and at least one second evacuation opening in a circumference of a wall of said evacuation flow path, wherein said second evacuation opening is located proximally to said first evacuation opening;
and wherein said at least one fluid evacuation flow path is configured to remove fluid and/or particles from said hollow organ through said at least one first evacuation opening and/or through said at least one second evacuation opening.

22-23. (canceled)

24. A system according to claim 21, comprising an outer sleeve surrounding said device, and wherein said at least one second evacuation opening is an opening in said outer sleeve or is in a fluid communication with at least one opening in said outer sleeve.

25. A system according to claim 1, wherein said at least one sideways washing opening has a maximal width or diameter in a range of 0.01 mm-2 mm.

26. A system according to claim 1, wherein said cryo inflow path and said washing inflow path are coaxial along at least 30% of a length of said washing inflow path.

27. A system according to claim 1, wherein said washing inflow path surrounds at least partly said cryo inflow path along at least 30% of a length of said cryo inflow path.

28. A system according to claim 57, wherein a surface of said optics assembly at said optics assembly distal end is curved or is angled relative to a long axis of the cryotherapy device, and wherein said at least one sideways washing opening is position, shaped and sized to release washing fluid towards said optics assembly surface.

29-56. (canceled)

57. A system according to claim 1, comprising an optics assembly having a distal end inside said elongated body configured to visualize a field of view between said device distal end and a target region inside said hollow organ, and wherein said at least one sideways washing opening is positioned in said wall of said washing inflow path to aim said washing fluid towards said optics assembly in said distal end and/or towards a field of view (FOV) distally to said optics assembly.
